(19) United States
(12) Patent Application Publication
(10) Pub. No.: US 2026/0298915 A1
(43) Pub. Date: Oct. 1, 2026

(54) A METHOD TO HOMOGENIZE THE CONCENTRATION OF BEADS ACROSS MULTIPLE LIQUID VOLUMES

(71) Applicant: NUCLERA LTD, Cambridge (GB)

(72) Inventors: Zachary GABER, Cambridge (GB); Sumit KALSI, Cambridge (GB); Johnathon LILLEY, Cambridge (GB); Andreas Michael WAEBER, Cambridge (GB)

(21) Appl. No.: 19/484,157

(22) PCT Filed: May 15, 2024
(86) PCT No.: PCT/GB2024/051261

§ 371 Date: Nov. 13, 2025

(30) Foreign Application Priority Data
May 15, 2023 (GB) 2307179.8

(51) Int. Cl. G01N 33/543; C07K 1/14; G01N 33/58

(52) U.S. Cl. CPC G01N 33/54326; C07K 1/14; G01N 33/582

(57) ABSTRACT

The invention provided herein relates to methods for normalising the concentration of beads in droplets on a microfluidic device, and methods for cell-free protein synthesis and characterisation of the expressed proteins.

### FIELD OF THE INVENTION

Provided herein are methods for normalising the concentration of beads in droplets on a microfluidic device, and methods for cell-free protein synthesis and characterisation of the expressed proteins.

### BACKGROUND

Proteins are biological macromolecules that maintain the structural and functional integrity of the cell, and many diseases are associated with protein malfunction. Protein purification is a fundamental step for analysing individual proteins and protein complexes and identifying interactions with other proteins, DNA or RNA. A variety of protein purification strategies exist to address desired scale, throughput and downstream applications. However, protein production can be challenging for many reasons. One major challenge is finding a suitable expression system, for example sourced from mammalian, bacterial, fungal, or plant cells. This can take months of work.

Cell-free protein synthesis (CFPS), also known as coupled or uncoupled in-vitro transcription and translation, is the production of peptides or proteins using biological machinery in a cell-free system, that is, without the use of living cells. The CFPS environment is not constrained within a cell wall or limited by conditions necessary to maintain cell viability, and enables the rapid production of any desired protein from a nucleic acid template, usually plasmid DNA or RNA from an in-vitro transcription. CFPS has been known for decades, and many commercial systems are available. Cell-free protein synthesis encompasses systems based on crude lysate (Cold Spring Harb Perspect Biol. 2016 December; 8(12): a023853) and systems based on reconstituted, purified molecular reagents, such as the PURE system for protein production (Methods Mol Biol. 2014; 1118: 275-284). CFPS requires significant concentrations of biomacromolecules, including DNA, RNA, proteins, polysaccharides, molecular crowding agents, and more (Febs Letters 2013, 2, 58, 261-268).

Split detectors such as split green fluorescent protein (GFP) systems are known for use in protein expression. A protein of interest having a GFP subcomponent can be detected by complementing with a detector species having the remainder of the GFP. Cabantous and Waldo describe such a system (In-vivo and in-vitro protein solubility assays using split GFP (NATURE METHODS|VOL. 3 NO. 10|OCTOBER 2006|845). The system described relies on expression in cells, from which the proteins of interest are lysed and then exposed to the detector in order to measure the level of expression.

WO2022/038353 describes cell-free expression of a protein having a GFP tag in the presence of a GFP detector species to measure signal as expression progresses.

Many proteins are expressed containing endogenous solubility factors. However factors such as for example maltose binding protein (MBP), Small Ubiquitin-like Modifier (SUMO), Glutathione S-transferase (GST) or thioredoxin (TRX) have a substantial size and may have undesirable effects on the proteins of interest to which they are attached. Additionally expression of large solubility elements uses the reagent resources during expression. Such sequences also take time to fold before acting at solubility enhancers, hence synthesising proteins of interest with solubility enhancing sequences attached thereto is far from ideal.

To date protein purification and analysis typically requires complex analysis techniques involving electrophoresis or requiring purified proteins. The inventors herein have developed simplified protein analysis and purification methods allowing multiple ways of characterising expressed proteins in their crude form.

One of the current challenges for cell-free protein synthesis is to increase the soluble yield of the expressed and purified proteins and to avoid aggregation or insolubility.

U.S. Pat. No. 8,809,068 describes methods for manipulating beads in microfluidic droplets. Methods involve ensuring beads are homogeneously distributed within individual droplets, but does not describe ways of ensuring different droplets contain the same number of particles.

WO2023079310 describes methods of purifying proteins on microfluidic devices. Methods involve manipulating beads within individual droplets, but does not describe ways of ensuring different droplets contain the same number of particles.

US20180243743 describes a device having magnetic conduits for manipulating beads in microfluidic droplets. Methods involve manipulating beads within individual droplets, but does not describe ways of ensuring different droplets contain the same number of particles.

WO2023/285821 describes efficient methods of splitting and manipulating droplets on microfluidic devices.

Purification of biomolecules can be performed using binding to beads. In order to compare whether purification steps have been effective for distinct molecular species the amount of beads needs to be homogenous between the different volumes containing reagents. Described herein are methods for improving the uniformity of bead dispersions between liquid volumes.

### SUMMARY OF THE INVENTION

In contrast to continuous flow microfluidics, interfacial forces play an important role in droplet microfluidics. The density of beads in the daughter droplets generated from a reservoir is affected by (1) the concentration and size of the beads, (2) the distribution of the beads in the reservoir at the time of droplet generation, (3) variability in the size of dispense or split, and (4) migration of beads from regions of high density to low density regions. Described herein are methods for improving the uniformity of bead dispersions between different liquid volumes. The bead volumes can be dispensed from larger reservoirs, for example to form droplets on a microfluidic device. Due to differences in the dispensed volumes or particle aggregation the number of beads can vary between different droplets. The inventors herein have appreciated that sequences of repeatedly pairwise merging and splitting of droplets interleaved with shuffling of the set of droplets allows a homogeneous dispersion where each droplet has the same concentration of suspended beads at the end of the merging and splitting process.

Described herein is a method for creating aqueous droplets having homogeneous concentrations of suspended beads wherein the process comprises:
a. taking a reservoir containing an aqueous liquid having beads suspended therein,
b. forming multiple droplet sub-volumes from the reservoir,
c. merging two or more of the droplets,
d. splitting the merged droplets, and
e. repeating steps c. and d. one or more times such that each of the final split droplets contains a homogeneous concentration of suspended beads.

Described herein is a method for creating aqueous droplets having homogeneous concentrations of suspended beads wherein the process comprises:
a. taking a reservoir containing an aqueous liquid having beads suspended therein,
b. forming multiple droplet sub-volumes from the reservoir,
c. merging two or more of the droplets,
d. splitting the merged droplets,
e. rearranging the set of droplets, and
f. repeating steps c., d. and e. one or more times such that each of the final split droplets contains a homogeneous concentration of suspended beads.

The step of rearranging droplets is helpful to counter biases introduced by environmental parameters such as non-uniform cell gaps or electrowetting force gradients. The pairwise merging and splitting in consecutive iterations does not involve the same droplet pairs. Whether by shuffling the droplets in between or by alternating merges with different neighbours, or a combination of the two, it is crucial that the same two droplets are not just merged and split over and over again. Thus the varying droplet pairs get to interact with other droplet pairs on the device. It is possible that each droplet formed from the reservoir can merge with and split from every other droplet formed from the reservoir.

For example 4 initial droplets A, B, C, D can be produced having a varying amount of beads in the 4 droplets. A/B and C/D are merged to form two droplets, A+B and C+D. The two droplets are split back into 4 droplets, A′, B′, C′, D′. A′ and B′ have a lower level of variation than A and B. C′ and D′ have a lower level of variation than C and D. In a second mixing A′/C′ and B′/D′ are mixed to form two droplets A′/C′ and B′/D′. These are split back into four droplets A″, B″, C″, D″. The positions of A″ and B″ may be switched such that A″ is next to D″ and B″ next to C″. In a third merging step, A″ can be mixed with D″ and B″ with C″. Following a third split, the concentrations of beads in the four final droplets should be more equal that the starting concentrations in A, B, C and D. The merging and splitting process can be repeated through multiple cycles to further improve homogeneity.

Alternatively the four droplets can be merged into a single droplet A+B+C+D, then split back into 4 droplets. The merging and splitting process can be repeated through multiple cycles to further improve homogeneity between all the split droplets.

Disclosed is a method that relies on parallel handling of volumes of fluid containing beads. The volumes of fluid may be less than 1 μL. The volumes of fluid may be less than 100 nL.

The beads may be made of any particular material. In some examples the beads may be magnetic. The beads may be non-magnetic.

Any number of droplets may be produced. For example 8 or more droplets may be produced. For example 16 or more droplets may be produced. For example 32 or more droplets may be produced. For 32 droplets, each droplet may be mixed with each of the other 31 droplets by a process of merging to form 16 droplets, splitting the 16 merged droplets, rotating a subset of the 32 droplets and repeating.

Any concentration of beads may be used. The method is advantageous where high bead concentrations are used as higher concentrations are more difficult to dispense homogeneously. The beads may be for example greater than 5% of the droplet volume. The beads may be 5-30% volume/volume. The beads may be greater than 20% volume/volume. The final dispensed homogenised concentration may be approximately 20% v/v. The final dispensed homogenised bead concentration may be 20% v/v+/−10% in each droplet.

The liquid volumes may be droplets on a digital microfluidic device, which may comprise active-matrix thin-film transistors.

The beads can be any particular size, for example greater than 1 μm in diameter. The beads may be greater than 10 μm in diameter. The beads may be greater than 25 μm in diameter. The beads may be magnetic beads of said dimensions.

The beads may be used to bind substances from the aqueous phase, for example to perform purification steps. The homogeneous bead droplets may be merged with droplets having substances which bind to the beads. The substances may be biomolecules such as nucleic acids or proteins.

The invention may include a method for determining the degree of aggregation of an expressed protein in a cell-free system comprising expressing a protein of interest (POI) in droplets on a digital microfluidic device, where the expressed POI is measured using a detectable signal and measuring the levels of soluble and insoluble POI. The level of aggregation can be used to determine whether a protein has expressed as soluble or insoluble. Soluble protein can be taken forward for purification, for example using magnetic beads. Soluble protein may be further purified, whereas proteins with a high degree of aggregation are unlikely to be purified or to be active.

Disclosed is a method for synthesising, characterising and purifying one or more proteins having detection tags and binding tags, the method comprising the steps of:
i mixing a cell-free protein expression system with one or more nucleic acid templates to form a plurality of merged liquid volumes; either
iia splitting the merged volumes into at least two aliquots and allowing protein expression in at least two aliquots; or
iib allowing protein expression then splitting into at least two aliquots;
iii adding a detector reagent which binds to the detection tags to at least one of the aliquots in order to determine the level of protein expression and determining the conditions where protein is expressed in soluble form;
iv selecting the volumes where protein has been solubly expressed and adding magnetic beads to a sub-population of the aliquots, wherein the beads bind to the binding tags and the beads in the droplets have been merged and split one or more times such that each of the final split droplets contains a homogeneous concentration of suspended beads;
v separating the magnetic beads from the expression reagents;
vi optionally adding wash reagents to the magnetic beads to remove unbound material;
vii eluting the bound protein from the magnetic beads by disrupting the binding to the binding tag; and
viii adding a detector reagent which binds to the detection tags to at least one of the eluted aliquots in order to determine the level of protein purification and determining the conditions where purified soluble protein is obtained.

The degree of aggregation can be measured directly, for example using light scattering, or may be seen using visible labels. The detection may be seen as optically detectable aggregates which cause light scatter, or may give rise to aggregates which can be detected by labelling, for example using fluorescence. The light scattering may be measured at an angle different from the incident light. The POI may have a binding sequence which binds to a detector moiety. The detector moiety can be added after the expression by combining additional droplets.

The degree of aggregation can be measured by counting the number of aggregates, the area of the aggregates, the intensity of the aggregates or by using a measurement of dispersion.

The POI may be expressed with a binding sequence which binds to a detector moiety. In which case a further droplet containing a detector moiety may be added to the droplet containing the POI to create an detectable signal in the combined droplet. The binding sequences may contain four or more amino acids. The binding sequences may contain 4-30 amino acids.

The detector moiety may be a protein. The detector moiety may comprise a component of a fluorescent protein such as for example sfGFP, or ccGFP.

The expressed protein may contain a sequence acting as a solubility enhancer, for example selected from:

| | |
|---|---|
| Glutathione S-Transferase | GST |
| Small Ubiquitin-like Modifier | SUMO |
| Maltose Binding Protein | MBP |
| Fasciola hepatica 8 kDa antigen | FH8 |
| Thioredoxin | TRX |
| Solubility Enhancing Ubiquitous Tag | SNUT |
| Seventeen kilodalton protein | SKP |
| Monomeric bacteriophage T7 orc protein | MOCR |
| E coli secreted protein A | ESPA |
| N-utilization substance | NusA |
| IgG domain B1 of Protein G | GB1 |
| IgG repeat domain ZZ of Protein A | ZZ |
| Mutated dehalogenase | HaloTag |
| Phage T7 protein kinase | T7PK |
| E. coli trypsin inhibitor | Ecotin |
| Calcium-binding protein | CaBP |
| Stress-response arsenate reductase | ArsC |
| N-terminal fragment of translation initiation factor IF2 | IF2-domain 1 |
| Stress-response protein | RpoA |
| Stress-response protein | SlyD |
| Stress-response protein | Tsf |
| Stress-response protein | RpoS |
| Stress-response protein | PotD |
| Stress-response protein | Crr |
| E. coli acidic protein | msyB |
| E. coli acidic protein | yjgD |
| E. coli acidic protein | rpoD |
| T7 phage tail | P17 |
| metal-binding protein | CUSF |
| 53-amino-acid-long N-terminal extension sequence | NEXT |

The detection tag may be one component of a fluorescent protein and the detector reagent a complementary portion of the fluorescent protein. The fluorescent protein could include sfGFP, ccGFP, GFP, eGFP, deGFP, frGFP, eYFP, eBFP, eCFP, Citrine, Venus, Cerulean, Dronpa, DsRED, mKate, mCherry, mRFP, FAST, SmURFP, miRFP670nano. For example the tag may be GFP₁₁ and the detector GFP₁₋₁₀. The tag may be one component of sfCherry. The tag may be sfCherry₁₁₈ and the detector sfCherry₁₋₁₀. The tag may be CFAST₁₁ or CFAST₁₀ and the detector NFAST in the presence of a hydroxybenzylidene rhodanine analog.

The tag may be ccGFP₁₁ and the detector ccGFP₁₋₁₀.

For example, the GFP₁₋₁₀ polypeptide amino acid sequence could be derived from sfGFP:

SEQ ID NO: 1
MSKGEELFTGVVPILVELDGDVNGHKFSVRGEGEGDATNGKLTLKFICT
TGKLPVPWPTLVTTLTYGVQCFSRYPDHMKRHDFFKSAMPEGYVQERTI
SFKDDGTYKTRAEVKFEGDTLVNRIELKGIDFKEDGNILGHKLEYNFNS
HNVYITADKQKNGIKANFKIRHNVEDGSVQLADHYQQNTPIGDGPVLLP
DNHYLSTQSVLSKDPNEK

Alternatively, the GFP₁₋₁₀ polypeptide amino acid sequence could be further mutated from the sequence above to become brighter more quickly upon complementation. The sequence may have a greater than 90% homology to any sequence mentioned herein. The sequence may have a greater than 95% homology to any sequence mentioned herein.

SEQ ID NO: 2
MSKGEELFTGVVPILVELDGDVNGHKFSVRGEGEGDATIGKLTLKFICT
TGKLPVPWPTLVTTLTYGVQCFSRYPDHMKRHDFFKSAMPEGYVQERTI
SFKDDGKYKTRAVVKFEGDTLVNRIELKGTDFKEDGNILGHKLEYNFNS
HNVYITADKQKNGIKANFTVRHNVEDGSVQLADHYQQNTPIGDGPVLLP
DNHYLSTQTVLSKDPNEK

The GFP₁₋₁₀ polypeptide amino acid sequence could also be derived from ccGFP, having a greater than 90% or 95% homology to:

(ccGFP₁₋₁₁)
SEQ ID NO: 3
MSLSKQVVKEDMKMTYHMDGCVNGHYFTIEGEGTGKPFKGQKTLKLRVTEGGPLPFAFDILSA
TFTYGNRCFCDYPEDMPDYFKQSLPEGYSWERTMMYEDGACGTASAHISLDKNGFVHNSTFH
GVNFPANGPVMKKKGVNWEPSSEKITACDGILKGDVTMFLVLEGGHRLKCLFQTTYKADKVVK
MPPNHIIEHRLVRSEDGDAVQIQEHAVAKYFTV
(ccGFP₁₋₁₀)
SEQ ID NO: 4
MSLSKQVVKEDMKMTYHMDGCVNGHYFTIEGEGTGKPFKGQKTLKLRVTEGGPLPFAFDILSA
TFTYGNRCFCDYPEDMPDYFKQSLPEGYSWERTMMYEDGACGTASAHISLDKNGFVHNSTFH
GVNFPANGPVMKKKGVNWEPSSEKITACDGILKGDVTMFLVLEGGHRLKCLFQTTYKADKVVK
MPPNHIIEHRLVRSED
(ccGFP₁₋₁₀)
SEQ ID NO 5
MSMEKQVLKENMKTTYHMDGSVDGHYFEIEGEGTGNPFKGEQELKLRVTKGGPLPFAFDILSP
TFTYGNRVFTDYPEDMPDYFKQSLPEGYSWERTMMYEDGATATASARISLDKNGFVHKSTFH
GENFPANGPVMKKKGVDWEPSSETITPEDGILKGDVEMFLVLEGGQRLKALFQTTYKANKVVK
MPPRHKIEHRLVRS
Nucleic acid sequence to express seq ID No 5 ccGFP₁₋₁₀;
SEQ ID NO: 6
5′atgagcatggaaaaacaggtgctgaaagaaaacatgaaaaccacctatcacatggatggtagcgttgatggtcactattttgaaat
tgaaggtgaaggcaccggcaatccgtttaaaggtgaacaagaactgaaactgcgtgttaccaaaggtggtccgctgccgtttgcattt
gatattctgagcccgacctttacctatggtaatcgtgtttttaccgactatccggaagatatgccggattatttcaaacagagcctgccg
gaaggttatagctgggaacgtaccatgatgtatgaagatggtgcaaccgcaaccgccagcgcacgtattagcctggataaaaatggtttt
gtgcataagagcacctttcacggtgaaaactttccggcaaatggtccggttatgaaaaagaaaggtgttgattgggaaccgagcagc
gaaaccattacaccggaagatggtattctgaaaggtgatgttgaaatgtttctggttctggaaggtggtcagcgtctgaaagccctgtttc
agaccacctataaagccaataaagtggttaaaatgcctccgcgtcataaaattgaacatcgtctggttcgtagc
SEQ ID NO 7
MSMSKQVLKENMKTTYHMDGSVNGHYFTIEGEGTGNPFKGQQSLKLRVTKGGPLPFAFDILSP
TFTYGNRVFTDYPEDMPDYFKQSLPEGYSWERTMMYEDGATATASARISLDKNGFVHKSTFH
GENFPANGPVMKKKGVNWEPSSETITPSDGILKGDVTMFLVLEGGQRLKALFQTTYKANKVVK
MPPRHKIEHRLVRS
Nucleic acid sequence to express seq ID No 7 ccGFP₁₋₁₀;
SEQ ID NO: 8
5′atgagcatgagcaaacaggtgctgaaagaaaatatgaaaaccacctatcacatggatggtagcgttaatggtcactattttaccatt
gaaggtgaaggcaccggtaatccgtttaaaggtcagcagagcctgaaactgcgtgttaccaaaggtggtccgctgccgtttgcatttg
atattctgagcccgacctttacctatggtaatcgtgtttttaccgactatccggaagatatgccggattatttcaaacagagcctgccgg
aaggttatagctgggaacgtaccatgatgtatgaagatggtgcaaccgcaaccgccagcgcacgtattagcctggataaaaatggttttgt
gcataagagcacctttcacggtgaaaactttccggcaaatggtccggttatgaaaaagaaaggtgttaattgggaaccgagcagcga
aaccattacaccgagtgatggtattctgaaaggtgatgttaccatgtttctggttctggaaggtggtcagcgtctgaaagccctgtttca
gaccacctataaagccaataaagtggttaaaatgcctccgcgtcataaaattgaacatcgtctggttcgtagc

The complementary GFP₁₁ peptide amino acid sequence could be the following:

1.
(SEQ ID NO: 9)
KRDHMVLLEFVTAAGITGT
2.
(SEQ ID NO: 10)
KRDHMVLHEFVTAAGITGT
3.
(SEQ ID NO: 11)
KRDHMVLHESVNAAGIT
4.
(SEQ ID NO: 12)
RDHMVLHEYVNAAGIT
5.
(SEQ ID NO: 13)
GDAVQIQEHAVAKYFTV
6.
(SEQ ID NO: 14)
GDTVQLQEHAVAKYFTV
7.
(SEQ ID NO: 15)
GETIQLQEHAVAKYFTE

or a truncated version thereof. Truncations may involve a shortening of up to 5 amino acids from the N terminus, the C terminus or a combination thereof.

GFP₁₁ or GFP₁₋₁₀ can be fused to the protein of interest through an amino acid linker. In one embodiment, the oligopeptide, peptide, or polypeptide linker can be 0-50 amino acids.

Also disclosed are nucleic acid sequences for expressing particular tags. Nucleic acid sequences include

SEQ ID NO: 16
5′GGTGATACCGTTCAGCTGCAAGAACATGCAGTTGCAAAATACTTTAC
CGTG
SEQ ID NO: 17
5′GGTGAAACCATCCAGTTACAAGAACACGCCGTGGCCAAATATTTCAC
CGAA

or a truncated version thereof.

These sequences may be repeated one or more times to produce a protein having multiple GFP₁₁ domains.

For example, the sfCherry₁₋₁₀ polypeptide amino acid sequence could be:

SEQ ID NO: 18
MEEDNMAIIKEFMRFKVHMEGSVNGHEFEIEGEGEGHPYEGTQTAKLKV
TKGGPLPFAWDILSPQFMYGSKAYVKHPADIPDYLKLSFPEGFTWERVM
NFEDGGVVTVTQDSSLQDGEFIYKVKLLGTNFPSDGPVMQKKTMGWEAS
TERMYPEDGALKGEINQRLKLKDGGHYDAEVKTTYKAKKPVQLPGAYNV
DIKLDITSHNED

The complementary sfCherry11 peptide amino acid sequence could be:

SEQ ID NO: 19
YTIVEQYERAEGRHSTGG

sfCherry11 or sfCherry₁₋₁₀ can be fused to the protein of interest through an amino acid linker. In one embodiment, the oligopeptide, peptide, or polypeptide linker can be 0-50 amino acids.

For example, the NFAST polypeptide amino acid sequence could be:

SEQ IS NO: 20
MEHVAFGSEDIENTLAKMDDGQLDGLAFGAIQLDGDGNILQYNAAEGDI
TGRDPKQVIGKNFFKDVAPGTDSPEFYGKFKEGVASGNLNTMFEWMIPT
SRGPTKVKVHMKKALS

The complementary CFAST₁₁ peptide amino acid sequence could be:

SEQ ID NO: 21
GDSYWVFVKRV

Or the complementary CFAST₁₀ peptide amino acid sequence could be:

SEQ ID NO: 22
GDSYWVFVKR

NFAST, CFAST₁₁, and/or CFAST₁₀ can be fused to the protein of interest through an amino acid linker. In one embodiment, the oligopeptide, peptide, or polypeptide linker can be 0-50 amino acids.

If desired the tag may be cleaved from the POI in order to remove the tag/detector. The cleavage may be performed for example using a protease or a metal cation.

The expression may be performed using cell-free protein synthesis reagents derived from whole cell extracts. The expression may be performed using cell-free protein synthesis reagents derived from reconstituted systems comprising assembled components for transcription and translation in a system of purified recombinant elements (PURE).

Any method described herein can be performed in for example microtitre plates or microcentrifuge tubes. Any method may be performed on a microfluidic or digital microfluidic device. The digital microfluidic device may comprise an oil-filled or humidified gaseous environment, wherein the humidified gaseous environment is achieved by enclosing or sealing the digital microfluidic device and providing on-board reagent reservoirs.

Disclosed is a method for expressing proteins in droplets on a digital microfluidic device having a two-dimensional array of planar microelectrodes wherein the proteins have a ccGFP₁₁ peptide amino sequence tag, wherein a portion of the droplets contain ccGFP₁₋₁₀ during the expression process and a further portion have ccGFP₁₋₁₀ added after expression and comparing the level of ccGFP₁₋₁₁ signal from the droplets.

The binding moiety for purification may contain four or more amino acids. The binding sequences may contain 4-30 amino acids. The binding moiety may be selected from:

Alfa-tag
(SEQ ID NO: 23)
(SRLEEELRRRLTE)
Avi-tag
(SEQ ID NO: 24)
(GLNDIFEAQKIEWHE)
C-tag
(SEQ ID NO: 25)
(EPEA)
Calmodulin-tag
(SEQ ID NO: 26)
(KRRWKKNFIAVSAANRFKKISSSGAL)
Dogtag
(SEQ ID NO: 27)
(DIPATYEFTDGKHYITNEPIPPK)
E-tag
(SEQ ID NO: 28)
(GAPVPYPDPLEPR)
FLAG
(SEQ ID NO: 29)
(DYKDDDDK)
G4T
(SEQ ID NO: 30)
(EELLSKNYHLENEVARLKK)
HA
(SEQ ID NO: 31)
(YPYDVPDYA)
His
(SEQ ID NO: 32)
(HHHHHH)
Isopeptag
(SEQ ID NO: 33)
(TDKDMTITFTNKKDAE)
lanthanide binding tag (LBT)
(SEQ ID NO: 34)
(FIDTNNDGWIEGDELLLEEG)
Myc
(SEQ ID NO: 35)
(EQKLISEEDL)
NE-Tag
(SEQ ID NO: 36)
(TKENPRSNQEESYDDNES)
Poly Glutamate-tag
(SEQ ID NO: 37)
(EEEEEEE)
Poly Arginine-tag
(SEQ ID NO: 38)
(RRRRRRR)
Rho1D4-tag
(SEQ ID NO: 39)
(TETSQVAPA)
SBP-tag
(SEQ ID NO: 40)
(MDEKTTGWRGGHVVEGLAGELEQLRARLEHHPQGQREP)
Sdytag
(SEQ ID NO: 41)
(DPIVMIDNDKPIT)
SH3
(SEQ ID NO: 42)
(STVPVAPPRRRRG)
SNAC
(SEQ ID NO: 43)
(GSHHW)
Snooptag
(SEQ ID NO: 44)
(KLGDIEFIKVNK)
Softag 1
(SEQ ID NO: 45)
(SLAELLNAGLGGS)
Softag 3
(SEQ ID NO: 46)
(TQDPSRVG)
Spot-tag
(SEQ ID NO: 47)
(PDRVRAVSHWSS)
Spytag
(SEQ ID NO: 48)
(AHIVMVDAYKPTK)
S-tag
(SEQ ID NO: 49)
(KETAAAKFERQHMDS)
Strep-tag
(SEQ ID NO: 50)
(AWAHPQPGG)
(SEQ ID NO: 51)
(AWRHPQFGG)
Strep-tag II
(SEQ ID NO: 52)
(WSHPQFEK)
T7tag
(SEQ ID NO: 53)
(MASMTGGQQMG)
TC-tag
(SEQ ID NO: 54)
(EVHTNQDPLD)
Ty-tag
(SEQ ID NO: 55)
(CCPGCC)
VSV-tag
(SEQ ID NO: 56)
(YTDIEMNRLGK)
Xpress-tag
(SEQ ID NO: 57)
(DLYDDDDK)

Disclosed is a method comprising expressing one or more proteins in one or more droplets on a digital microfluidic device having a two-dimensional array of planar microelectrodes, wherein the proteins have a ccGFP₁₁ peptide amino sequence tag and a Strep-tag, splitting the droplets into at least two volumes, merging droplets containing ccGFP₁₋₁₀ into one of the droplets following the expression process and measuring the uniformity of the fluorescent signal to determine the degree of aggregation of the expressed protein, taking corresponding droplets without ccGFP₁₋₁₀ which contain protein having been solubly expressed and adding magnetic beads which bind to the strep-tag, wherein the droplets having the magnetic beads have been merged and split one or more times such that each of the final split droplets contains a homogeneous concentration of suspended beads, immobilizing and washing the beads to remove unbound material; eluting the POI by disrupting the strep binding, adding droplets containing ccGFP₁₋₁₀ following the elution process and measuring the fluorescent signal in order to determine the level of protein purification and determining the conditions where purified soluble protein is obtained.

The method may be performed on different sequences in parallel. The method may use at least 8 different nucleic acid templates, which may be screened against at least 4 different expression reagents on the same device. The device may be capable of handling many droplets in parallel, for example the device may separately manipulate at least 192 droplets.

The expression comparison and purification screen can identify the optimal conditions for expression and purification of the desired protein in its most soluble and stable form.

### FIGURES

FIG. 1 shows a protein of interest with a peptide tag (detection tag) in the presence of a protein that binds to the peptide tag (detector protein). In the case that the detection tag and detector protein generate a detectable signal upon binding, then the quantity of the protein of interest can be measured.

FIG. 2 shows a cartoon representation of protein expression of different proteins having varying soluble yields and levels of aggregation. Some conditions give no expression (dark). Some conditions give a high level of soluble protein (uniform intensity white squares). Some conditions give protein aggregates, meaning the expressed proteins are insoluble and clump together (white spots in dark background). Some give a mix of soluble and aggregated proteins (white spots in a grey background). Thus the level of soluble expression and level of aggregation can be determined by adding a detector species to the expressed proteins.

FIG. 3 shows a schematic workflow for protein expression. DNA linear expression constructs (LEC) can be mixed with expression reagents and merged. The mixed reaction can be split into multiple aliquots and left to undergo protein expression. A soluble detector species (DET) is added to one drop after expression has occurred. Where the expressed protein is soluble and the degree of aggregation is low, the signal is uniform, showing the protein is solubly expressed.

FIG. 4 shows a schematic workflow for protein expression. DNA linear expression constructs (LEC) can be mixed with expression reagents and merged. The mixed reaction can be split into multiple aliquots and left to undergo protein expression. A soluble detector species (DET) is added to one drop after expression has occurred. Where the expressed protein is aggregated, the detector gives a non-uniform signal, indicating the expressed protein is to some degree insoluble or aggregated. The insoluble or aggregated POI is not likely to be purified from the droplets.

FIG. 5 shows the experimental result from 24 different proteins expressed in a reconstituted cell-free protein synthesis system in droplets on an electrowetting on dielectric (EWoD) device. Each construct contains a GFP₁₁ tag. In the rows marked Screen, the GFP₁₋₁₀ detector species is present from the start of expression. The rows marked Endpoint shows the fluorescence signal from 10 hours expression in the absence of GFP₁₋₁₀ detector species followed by 5 hours complementation with the GFP₁₋₁₀ detector species. This experiment showed significant differences between expression/complementation in Screen Biolnk compared to Endpoint detection. The detected protein clusters formed after expression only with endpoint detection mean that the protein aggregated after expression, thereby lowering the soluble yield. It is evident from the image the presence of speckles for several constructs, indicating the likely presence of protein aggregates. The level of aggregation enables identification of conditions which are worthy of further testing, and identification of conditions having high level of aggregated protein from which further purification is unlikely to give material.

FIG. 6 shows a schematic workflow for protein expression and purification. DNA linear expression constructs (LEC) can be mixed with expression reagents and merged. The mixed reaction can be split into multiple aliquots and left to undergo protein expression. A soluble detector species (DET) is added to one drop after expression has occurred. Where the expressed protein is aggregated, the detector gives a non-uniform signal, indicating the expressed protein is to some degree insoluble or aggregated. The insoluble or aggregated POI is not likely to be purified from the droplets, and such droplets are not taken for purification. Where the expressed protein is soluble and the degree of aggregation is low, the signal is uniform, showing the protein is solubly expressed. Such droplets, or a subset thereof are taken for purification. Droplets containing beads can be added in order to capture the expressed POI having the binding tags. The beads can be immobilised and washed by adding additional droplets and immobilizing the beads whilst the droplets are removed. The bound material can be eluted from the beads by disrupting the binding. The process of addition of detector can be repeated. Where a high signal is seen from soluble material, the protein has been purified and eluted. Where the signal is low, absent or aggregated, limited soluble purified POI has been obtained. The results from detector screen 1 and 2 can be compared in order to establish conditions for expression and purification.

FIG. 7 shows a cartoon schematic of the workflow in FIG. 6. After the optimal conditions have been identified, the protein can be produced using a scale up to obtain a larger volume of material. For example 24 DNA constructs can be screened in 8 different conditions to determine the best expression. The 24 constructs may be 24 independent sequences for different POIs or may be a single POI with 24 varying flanks (solubility tags etc). The 8 conditions may be lysates or reconstituted systems have different components. After identification of the best expression systems, reagents can be selected for a purification screen. The screen may select for example 30 liquid volumes (droplets) to which beads are added. The conditions giving the best purification can be scaled up in tubes off device to allow soluble proteins to be obtained.

FIG. 8 shows an expression screen for protein VEGF with varying flank sequences. An expression and solubility screen was first performed to determine the optimal expression of human VEGF using three different cell-free reagents in combination with constructs containing 8 different nucleic acid template variations corresponding to VEGF with various solubility tags in the N- and C-terminus. These three cell-free blends consist of different cell-free protein synthesis reagents. Blend-3 is designed to promote disulfide bond formation while Blend-4 contains chaperones that promote protein folding. Blend-2 contains the core component of the cell-free synthesis reagents without any additional components. Each construct was run in duplicate. 192 droplets were run in total on a single cartridge. The dotted lines denote the cell-free blends run with the corresponding constructs. The position of the detector (DET) is also noted. Bright white fluorescent spots indicate expression of soluble proteins.

FIG. 9 shows a graphical representation of spot intensity in FIG. 8. Concentration of proteins expressed is calculated based on fluorescent intensity measured. Construct A5 (SUMO_VEGF_DET) produced the highest expression in Blend-3. Constructs with the highest yield (mg/mL) were selected for on-cartridge purification (highlighted in green dashed box).

FIG. 10 shows an image of thirty soluble candidates that presented the highest expression yield which were automatically selected and taken forward for an on-cartridge purification step. Among these candidates were SUMO-VEGF expressed in Blend-2, Blend-3 and Blend-4. These expression candidates were purified on the cartridge using magnetic beads. FIG. 5 shows the location of SUMO_VEGF_STREP_DET purified on the cartridge. Fluorescent intensity measurement was used to determine yield of the purified proteins.

FIG. 11 shows a graphical representation of FIG. 10 and shows a side by side comparison of the expression yield and purified yield for SUMO-VEGF-STREP_DET. The results show that this construct, expressed in all three cell-free blends, is soluble and can be purified. Blend-3 yields the highest protein expression and purified yield and therefore was chosen for off-cartridge scale up expression.

FIG. 12 shows a gel showing off cartridge, scaled-up expression (500 μL CFPS reaction) and purification of SUMO_VEGF_STREP_DET. SDS-PAGE illustrates the VEGF protein with a molecular weight slightly higher than the predictive weight of 36.32 kDa as the SUMO tag normally shows higher than its calculated molecular weight on an SDS-PAGE gel. Lane 1 molecular weight ladder, lane 2 Blend-3 negative control, lane 3 CFPS total after expression, lane 4 CPFS (soluble) obtained following centrifugation and soluble fraction obtained, lane 5 Unbound; flow through sample not bound to beads, lane 6 Purified (elute); sample eluted off of the resin bead.

FIG. 13 shows an 8×4 droplet array with droplet repositioning following each cycle. Magnetic particles were visualized and quantified via fluorescent imaging of fluorescein bound to the surface of the magnetic particle. The image shows 2 devices having an array of 32× droplets containing magnetic bead microparticles dispensed in 49 px² droplets in an 8×4 formation.

FIG. 14 shows the rearrangement of droplets to give homogeneous mixing. Each array undergoes a routine as shown for a number of cycles (40 in this case) where magnetic bead containing droplets are mixed and split continuously.

FIG. 15 shows the fluorescent image of the two arrays following the cycling.

FIG. 16 shows a representation of the mixing process for the top 8 droplets.

FIG. 17 shows the distribution of bead pellet size with and without homogenisation. The bead pellet size indicates the number of beads in each droplet as the beads are pinned to the surface.

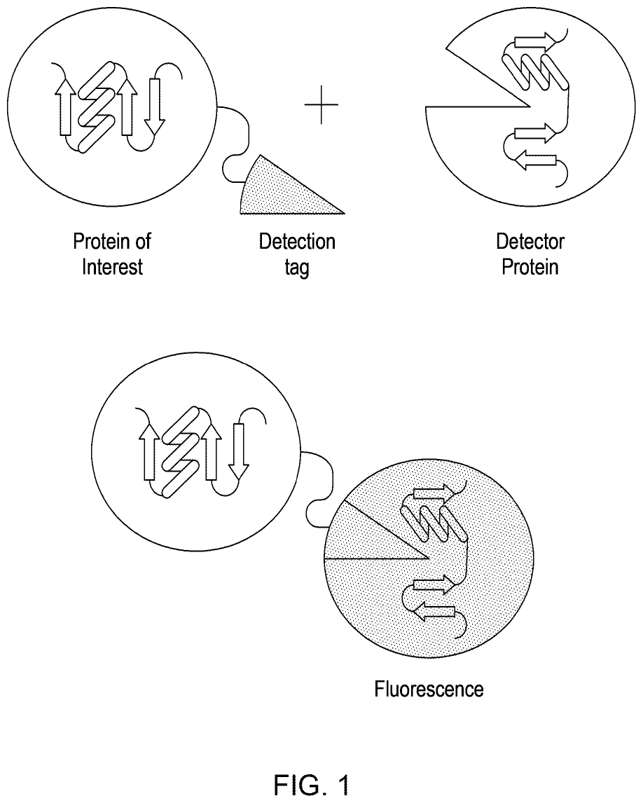

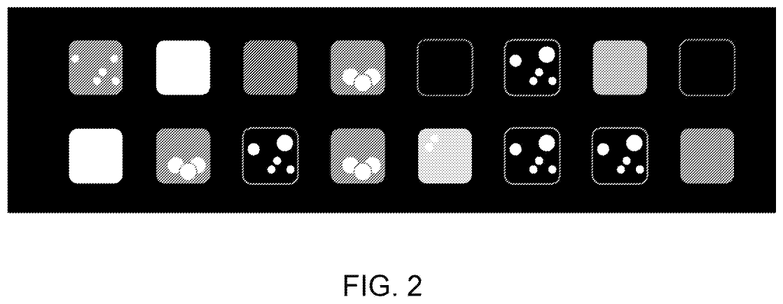

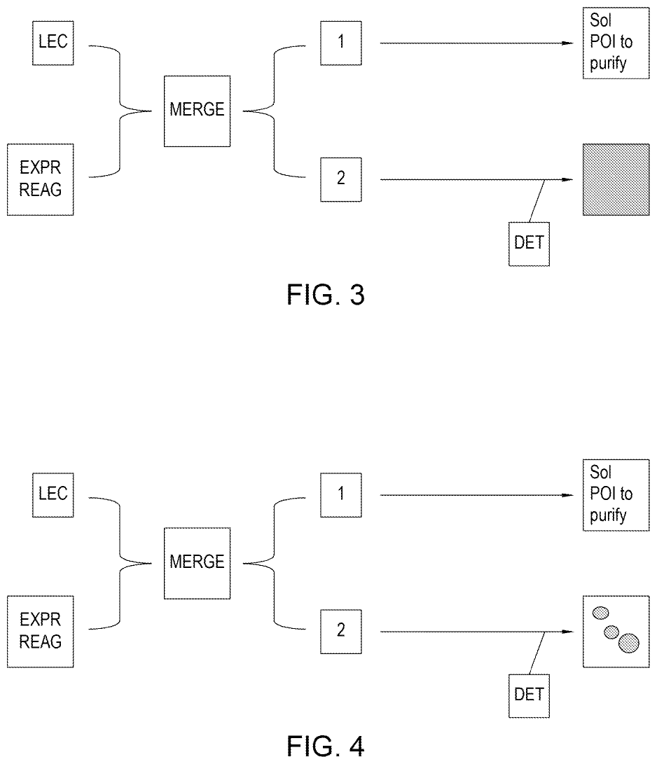

### DETAILED DESCRIPTION OF THE INVENTION

Applicants have appreciated that the purification of protein sequences is not predictable and requires screening. Just because a protein is expressed, does not mean it can be obtained in a soluble purified form. The level of aggregation following protein expression is an important metric in determining whether a protein can be subsequently purified. A high level of protein aggregation may indicate a lack of soluble protein for purification. Disclosed herein is a method for determining the degree of aggregation of an expressed protein in a cell-free system comprising expressing a protein of interest (POI) in droplets on a digital microfluidic device, where the expressed POI is measured by an optically detectable signal and measuring the levels of insoluble POI.

In order to accurately measure and compare the amounts of purified proteins, large numbers of droplet volumes having the same amount of beads are required. A lack of homogeneity in the inter-droplet bead distribution introduces measurement errors in the amount of purified protein which are unrelated to the starting protein concentration. Assays developed by the inventors require bead homogeneity across multiple droplets.

Inhomogeneous distributions can be normalised by repeated mixing and splitting. For example electrowetting control allows two or more droplets to be merged and split. If all droplets are merged with and split from other droplets, any differences between the initial droplet concentrations are reduced or removed. Such homogeneous droplets can be used in assays to measure the success of purification without concerns regarding a lack of starting homogeneity in the beads used for purification.

Disclosed is a method that relies on parallel handling of volumes of fluid. Described herein is a method for creating aqueous droplets having homogeneous concentrations of suspended beads wherein the process comprises:
a. taking a reservoir containing an aqueous liquid having beads suspended therein,
b. forming multiple droplet sub-volumes from the reservoir,
c. merging two or more of the droplets,
d. splitting the merged droplets, and
e. repeating steps c. and d. one or more times such that each of the final split droplets contains a homogeneous concentration of suspended beads.

Described herein is a method for creating aqueous droplets having homogeneous concentrations of suspended beads wherein the process comprises:
a. taking a reservoir containing an aqueous liquid having beads suspended therein,
b. forming at least four droplet sub-volumes from the reservoir,
c. merging each of the droplets with at least another droplet,
d. splitting the merged droplets to form at least 4 further droplets, and
e. repeating steps c. and d. one or more times such that each of the final split droplets contains a homogeneous concentration of suspended beads.

Disclosed is a method for synthesising, characterising and purifying one or more proteins having detection tags and binding tags, the method comprising the steps of:
i mixing a cell-free protein expression system with one or more nucleic acid templates to form a plurality of merged liquid volumes; either
iia splitting the merged volumes into at least two aliquots and allowing protein expression in at least two aliquots; or
iib allowing protein expression then splitting into at least two aliquots;
iii adding a detector reagent which binds to the detection tags to at least one of the aliquots in order to determine the level of protein expression and determining the conditions where protein is expressed in soluble form;
iv selecting the volumes where protein has been solubly expressed and adding magnetic beads to a sub-population of the aliquots, wherein the beads bind to the binding tags and the beads in the droplets have been merged and split one or more times such that each of the final split droplets contains a homogeneous concentration of suspended beads;
v separating the magnetic beads from the expression reagents;
vi optionally adding wash reagents to the magnetic beads to remove unbound material;
vii eluting the bound protein from the magnetic beads by disrupting the binding to the binding tag; and
viii adding a detector reagent which binds to the detection tags to at least one of the eluted aliquots in order to determine the level of protein purification and determining the conditions where purified soluble protein is obtained.

When expressing proteins, various parameters are important for determining successful expression, both in terms of yield and function. One of the factors is the soluble yield of expressed proteins, as many proteins express in forms that are insoluble or become inactive through a lack of stability.

The assays for soluble yield described herein involve expression and detection of the expressed proteins to measure the degree of aggregation. The protein of interest (POI) may have a detection tag which binds to a detector to generate a signal. The detector are/or POI may have a further solubility factor.

The method may be used as part of a process to determine whether an expressed protein is suitable for subsequent purification testing. For example the method may involve expressing a protein of interest (POI) in a first reagent volume and splitting the reagent volume into multiple aliquots. The splitting may be performed before or after the expression process. The protein of interest may have a detector tag which complements a detector species and becomes fluorescent. The detector species is added to one or more of the aliquots and the signal recorded. The uniformity of the signal in the droplet determines the degree of aggregation. Where the signal is homogenous across the droplet, the POI is soluble, with little or no aggregation. Where the signal is non-homogenous across the droplet, seen in the form of clumps, the protein is aggregated and insoluble. Thus the level of overall signal indicates the level of expression and the signal uniformity determines aggregation. Thus the level of expression, and the level of soluble expression can be determined from looking at the amount of, and uniformity of the signal within the droplet. The degree of aggregation may be measured by using a measurement of uniformity, counting the number of aggregates, the area of the aggregates, the intensity of the aggregates or by using a measurement of dispersion.

Protein expression may give rise to aggregation if the proteins are unstable or poorly soluble. Protein aggregates give rise to a lack of homogeneous signal from the detector. Thus POIs having a high soluble yield give a uniform level of signal, proteins having a low soluble yield may have either a low or high level of signal, but the signal aggregates and is not homogenous throughout the reaction volume as the proteins are aggregated.

Assays for protein stability and potential for further purification may be performed on the device. Further assays for protein stability may include
Solubility
Aggregation (over time)
Thermal Stability

Protein thermal stability does not necessarily predict aggregation rate at low temperature (and vice versa). For example, the melting temperature (Tm) of a monoclonal antibody does not necessarily correlate to the aggregation rate of said monoclonal antibody at room temperature (Mol. Pharmaceutics 2016, 13, 307-319). At high temperatures, the aggregation rate is dependent on the unfolded state whereas at low temperature, the aggregation rate is dependent on the native state. Thus denaturing a protein may or may not give an accurate measure of stability for the protein in its native state.

Providing a protein aggregation metric over time would be highly useful and present additional information in addition to simply a measurement of the expressed yield. Producing stable protein formulations is key to the development and manufacturing of proteins. The robustness of the formulations against external stress factors is crucial as proteins may be exposed to various types of stress such as temperature, pH, salts, mechanical stress, surface interaction or oxidation. Protein aggregation is a common issue faced during expression. Therefore, the prediction or control of protein aggregation during the production of a protein of interest is much needed. Described herein is a high throughput assay for protein aggregation/stability which measures the protein stability using the directly expressed material.

Protein purification can be challenging and time consuming, often resulting in a significant loss of material or a failure to obtain material in purified form. Therefore it is desirable to measure protein stability directly after expression before having to purify the proteins from the proteins used in expression. Measurement in crude cell lysates or directly in cell-free expression systems are possible using optical based analysis techniques, as these can tolerate the presence of other proteins which are stable to the conditions being measured or are not otherwise detectable.

Disclosed is a method for protein synthesis comprising expressing a protein, wherein the expressed protein contains a sub-component of a fluorescent protein, the method comprising monitoring stability of the protein over time by creating the fluorescent protein in different batches of material at different times such as before and after purification.

The method for measuring stability avoids the need for complex purification steps or gel based separations prior to performing the assay. A fluorescence based assay can be used to measure the amount of expressed protein (soluble yield) in conjunction measuring protein stability. Thus both yield and stability can be determined after expression without needing to run gels or purify material.

The fluorescence measurement can act as a real time measure of soluble protein expression. As the tag sequence is produced, the presence of the remaining fluorescent protein as a detector species then allows real-time measurement of the tag sequence and the amount of soluble protein. Alternatively the fluorescence can be measured by adding the detector species after expression to measure insoluble protein. The fluorescence can be retained and monitored during the stability assay. Alternatively further detector protein can be added after the stability step to determine the remaining protein level. Both yield of synthesis and stability of expressed protein can thus be determined. For example GFP₁₁ can be attached to the expressed protein and GFP₁₋₁₀ used as the detector species.

This screening workflow enables users to rapidly screen different expression systems in the form cell-free lysates, or reconstituted systems. Having identified an optimal expression system, soluble yield, stability and purification conditions can be measured directly on the same device.

The protein can be expressed with a binding tag. The binding moiety can be a region of amino acid/peptide sequence. The affinity binding site can be a region of amino acid/peptide sequences specific to a particular antibody. The tag can be attached to the N or C terminus. For example the binding moiety can be selected from the list of exemplary peptide affinity binding sites below:

Alfa-tag
(SEQ ID NO: 23)
(SRLEEELRRRLTE)
Avi-tag
(SEQ ID NO: 24)
(GLNDIFEAQKIEWHE)
C-tag
(SEQ ID NO: 25)
(EPEA)
Calmodulin-tag
(SEQ ID NO: 26)
(KRRWKKNFIAVSAANRFKKISSSGAL)
Dogtag
(SEQ ID NO: 27)
(DIPATYEFTDGKHYITNEPIPPK)
E-tag
(SEQ ID NO: 28)
(GAPVPYPDPLEPR)
FLAG
(SEQ ID NO: 29)
(DYKDDDDK)
G4T
(SEQ ID NO: 30)
(EELLSKNYHLENEVARLKK)
HA
(SEQ ID NO: 31)
(YPYDVPDYA)
His
(SEQ ID NO: 32)
(HHHHHH)
Isopeptag
(SEQ ID NO: 33)
(TDKDMTITFTNKKDAE)
lanthanide binding tag (LBT)
(SEQ ID NO: 34)
(FIDTNNDGWIEGDELLLEEG)
Myc
(SEQ ID NO: 35)
(EQKLISEEDL)
NE-Tag
(SEQ ID NO: 36)
(TKENPRSNQEESYDDNES)
Poly Glutamate-tag
(SEQ ID NO: 37)
(EEEEEEE)
Poly Arginine-tag
(SEQ ID NO: 38)
(RRRRRRR)
Rho1D4-tag
(SEQ ID NO: 39)
(TETSQVAPA)
SBP-tag
(SEQ ID NO: 40)
(MDEKTTGWRGGHVVEGLAGELEQLRARLEHHPQGQREP)
Sdytag
(SEQ ID NO: 41)
(DPIVMIDNDKPIT)
SH3
(SEQ ID NO: 42)
(STVPVAPPRRRRG)
SNAC
(SEQ ID NO: 43)
(GSHHW)
Snooptag
(SEQ ID NO: 44)
(KLGDIEFIKVNK)
Softag 1
(SEQ ID NO: 45)
(SLAELLNAGLGGS)
Softag 3
(SEQ ID NO: 46)
(TQDPSRVG)
Spot-tag
(SEQ ID NO: 47)
(PDRVRAVSHWSS)
Spytag
(SEQ ID NO: 48)
(AHIVMVDAYKPTK)
S-tag
(SEQ ID NO: 49)
(KETAAAKFERQHMDS)
Strep-tag
(SEQ ID NO: 50)
(AWAHPQPGG)
(SEQ ID NO: 51)
(AWRHPQFGG)
Strep-tag II
(SEQ ID NO: 52)
(WSHPQFEK)
T7tag
(SEQ ID NO: 53)
(MASMTGGQQMG)
TC-tag
(SEQ ID NO: 54)
(EVHTNQDPLD)
Ty-tag
(SEQ ID NO: 55)
(CCPGCC)
VSV-tag
(SEQ ID NO: 56)
(YTDIEMNRLGK)
Xpress-tag
(SEQ ID NO: 57)
(DLYDDDDK)

The binding moiety may include a small molecule affinity tag such as biotin. The binding moiety may include a particular sequence of nucleic acids.

The expressed amino acid sequences can be bound to beads having an affinity to the tag. Suitable beads may be magnetic or paramagnetic beads. The beads may have for example metal ions to chelate to poly His or a streptavidin or modified streptavidin to bind to the strep-tag. The beads may be of a size that can be handled within a droplet, for example 1-50 μM in average diameter. The beads may have a magnetic core and a polymer coating. The beads may be for example silica or agarose. Suitable beads are commercially available.

The beads can be merged into droplets on the device. When exposed to a magnetic field, the beads can be immobilised whilst the droplets can be moved or split. Thus the beads can be removed from the unbound reagents. Washing steps can be performed by adding further reagents, agitating the beads in the droplets, immobilising the beads and removing the droplet.

The binding can be reversed using suitable buffers, for example containing biotin to release a strep-tag or imidazole to release poly-his.

The process can be performed in droplets, which can be manipulated by electrokinesis in order to effect and improve protein expression and analysis. The droplet can be moved using any means of electrokinesis. The droplet can be moved using electrowetting on dielectric (EWoD). The electrical signal on the EWoD or optical EWoD device can be delivered through segmented electrodes, active-matrix thin-film transistors, or digital micromirrors.

The cell-free expression of peptides or proteins can use a cell lysate having the reagents to enable protein expression or a reconstituted system. Common components of a cell-free reaction include an energy source, a supply of amino acids, cofactors such as magnesium, and the relevant enzymes. A cell extract is obtained by lysing the cells of interest and removing the cell walls, DNA genome, and other debris by centrifugation. The remains are the cell machinery including ribosomes, aminoacyl-tRNA synthetases, translation initiation and elongation factors, nucleases, etc. Once a suitable nucleic acid template is added, the nucleic acid template can be expressed as a peptide or protein using the cell derived expression machinery.

In order to optimise expression, the expression system can be supplemented with additional components, including purified enzymes. The additional components may include salts, co-factors, buffers, surfactants, chaperones or additional protein components. The additional protein components may be selected from for example chaperones, glycosylating enzymes, proteases, redox active enzymes, phosphorylases and kinases.

The expression composition may be assembled on the device from mixing a variety of droplets in order to screen a variety of compositions in parallel.

By way of example, the screening reagents may include,
Chaperone mix (e.g., PUREfrex GroE mix)
Kinase 1 (e.g., NEB CK2)
Kinase 2 (e.g., NEB PKA)
Protease 1 (e.g., NEB TEV)
Protease 2 (e.g., Merck HRV 3C)
Common metal ions cocktail
Common co-factors cocktail

The compositions can be blended by the user and the level of expression of the protein of interest monitored in each of the blended conditions.

Any particular nucleic acid template can be expressed using the system described herein. Three types of nucleic acid templates used in cell-free protein synthesis (CFPS) include plasmids, linear expression constructs (LECs), and mRNA. Plasmids are circular templates, which can be produced either in cells or synthetically. LECs can be made via PCR. mRNA can be produced through in-vitro transcription systems. The methods can use a single nucleic acid template per droplet. The methods can use multiple nucleic acid templates per drop. The methods can use multiple droplets having a different nucleic acid template per droplet.

An energy source is an important part of a cell-free reaction. Usually, a separate mixture containing the needed energy source, along with a supply of amino acids, is added to the extract for the reaction. Common sources are phosphoenolpyruvate, acetyl phosphate, and creatine phosphate. The energy source can be replenished during the expression process by adding further reagents to the droplet during the process.

The cell-free extract having the components for protein expression includes everything required for protein expression apart from the nucleic acid template. Thus the term includes all the relevant ribosomes, enzymes, initiation factors, nucleotide monomers, amino acid monomers, metal ions and energy sources. Once the nucleic acid template is added, protein expression is initiated without further reagents being required.

Thus the cell-lysate can be supplemented with additional reagents prior to the template being added. The cell-free extract having the components for protein expression would typically be produced as a bulk reagent or ‘master mix’ which can be formulated into many identical droplets prior to the distinct template being separately added to separate droplets. Common cell extracts in use today are made from E. coli (ECE), rabbit reticulocytes (RRL), wheat germ (WGE), insect cells (ICE) and Yeast Kluyveromyces (the D2P system). All of these extracts are commercially available.

Rather than originating from a cell extract, the cell-free system can be assembled from the required reagents. Systems based on reconstituted, purified molecular reagents are commercially available, for example the PURE system for protein production, and can be used as supplied. The PURE system is composed of all the enzymes that are involved in transcription and translation, as well as highly purified 70S ribosomes. The protein synthesis reaction of the PURE system lacks proteases and ribonucleases, which are often present as undesired molecules in cell extracts.

The use of a population of droplets having different components allows the rapid screening of a variety of variable factors to identify optimal conditions for expression of a desired proteins. The protein can be contained with a sequence having other amino acid domains, for example solubility factors or binding tags.

The flank sequences attached to the target sequence may include elements required for transcription and translation, such as for example a promoter region such as a T7 promoter binding site, a ribosome binding site, start codons and stop codons. The flank sequences may optionally also include elements such as solubility tags, purification tags or detection tags.

Protein solubility sequences may be attached to the detector and the POI sequences. For example the GFP₁₋₁₀ may be attached to further elements to improve solubility. The solubility enhancing sequence may be a peptide sequence or a naturally occurring sequence. The solubility enhancing sequence may be selected from for example maltose binding protein (MBP), Small Ubiquitin-like Modifier (SUMO), Glutathione S-transferase (GST) or thioredoxin (TRX). The tags may be attached to either the C or N terminus. Any example of a solubility enhancer may be used. A list of possible proteins is shown below. Any sequence selected from the list below may be chosen:

| | |
|---|---|
| Glutathione S-Transferase | GST |
| Small Ubiquitin-like Modifier | SUMO |
| Maltose Binding Protein | MBP |
| Fasciola hepatica 8 kDa antigen | FH8 |
| Thioredoxin | TRX |
| Solubility Enhancing Ubiquitous Tag | SNUT |
| Seventeen kilodalton protein | SKP |
| Monomeric bacteriophage T7 orc protein | MOCR |
| E coli secreted protein A | ESPA |
| N-utilization substance | NusA |
| IgG domain B1 of Protein G | GB1 |
| IgG repeat domain ZZ of Protein A | ZZ |
| Mutated dehalogenase | HaloTag |
| Phage T7 protein kinase | T7PK |
| E. coli trypsin inhibitor | Ecotin |
| Calcium-binding protein | CaBP |
| Stress-response arsenate reductase | ArsC |
| N-terminal fragment of translation initiation factor IF2 | IF2-domain 1 |
| Stress-response protein | RpoA |
| Stress-response protein | SlyD |
| Stress-response protein | Tsf |
| Stress-response protein | RpoS |
| Stress-response protein | PotD |
| Stress-response protein | Crr |
| E. coli acidic protein | msyB |
| E. coli acidic protein | yjgD |
| E. coli acidic protein | rpoD |
| T7 phage tail | P17 |
| metal-binding protein | CUSF |
| 53-amino-acid-long N-terminal extension sequence | NEXT |

Any fluorescent protein may be used. The term GFP is used herein to describe a group of green fluorescent proteins from different organisms including the proteins sfGFP, GFP, eGFP, ccGFP, deGFP, frGFP. The fluorescent protein may be sfGFP, GFP, eGFP, ccGFP, deGFP, frGFP, eYFP, eBFP, eCFP, Citrine, Venus, Cerulean, Dronpa, DsRED, mKate, mCherry, mRFP, FAST, SmURFP, miRFP670nano. For example the peptide tag may be GFP₁₁ and the further polypeptide GFP₁₋₁₀. The peptide tag may be one component of sfCherry. The peptide tag may be sfCherry₁₁ and the further polypeptide sfCherry₁₋₁₀. The peptide tag may be CFAST₁₁ or CFAST₁₀ and the further polypeptide CFAST in the presence of a hydroxybenzylidene rhodanine analog. The peptide tag may be ccGFP₁₁ and the further polypeptide ccGFP₁₋₁₀.

The fluorescent protein may be GFP. The fluorescent protein may be sfGFP. The fluorescent protein may be ccGFP. The solubility enhancement moiety may comprise ccGFP₁₋₁₀ and MBP.

The protein may be assembled and thereby become fluorescent as a result of the expressed protein binding with the binding partner. The affinity interaction results in the two sub-components of the fluorescent protein being near enough to each other to bind and induce fluorescence.

The complementary GFP₁₁ peptide amino acid sequence tag could be the following:

1.
KRDHMVLLEFVTAAGITGT
2.
KRDHMVLHEFVTAAGITGT
3.
KRDHMVLHESVNAAGIT
4.
RDHMVLHEYVNAAGIT
5.
GDAVQIQEHAVAKYFTV
6.
GDTVQLQEHAVAKYFTV
7.
GETIQLQEHAVAKYFTE

Or a truncated sequence thereof. or a truncated version thereof. Truncations may involve a shortening of up to 5 amino acids from the N terminus, the C terminus or a combination thereof.

Properties of the expressed protein may be characterised on the device. An initial screen may be based on the level of soluble expression by measuring fluorescence formed on complementation of a detector with the expressed sequence. The protein may remain fluorescent during immobilisation, at which point the level of affinity purification can be determined. Alternatively a non-fluorescent portion of the protein can be purified, and the detector added to measure the soluble yield of the purified material.

Disclosed is a method of taking multiple droplets having a protein with a GFP₁₁ tag and monitoring changes in the level of the GFP₁₁ tag in order to determine protein expression yield.

The methods can measure aggregation by measuring solubility before and after purification. For example,
1. A mother droplet containing nucleic acid templates and protein expression reagents expresses the POI.
2. The mother droplet is split into multiple daughter droplets (either before, during or after expression).
3. A droplet containing detector is added to daughter droplet 1.
4. A subset of the daughter 2 droplets is chosen to have beads added, binding the expressed protein.
5. The beads are washed to remove unbound material.
6. The POI is removed from the beads.
7. Another droplet containing detector is added to the eluted POI.
8. The complementations from (7) and (3) are run for the same volume and period of time each.
9. The fluorescence is measured from both droplets and compared. Protein soluble yield is measured from the pre and post purification in order to determine purification yield.

Once the protein is expressed the expression can be stopped by addition of chemical reagents to prevent further expression. The assay for purification should be independent of further expression, as increases in the amount of protein by further protein expression may bias the measurements of purification. For example the detector solution can be mixed with an agent that arrests protein synthesis, such as a Mg++ chelator (EDTA) in order to arrest and detect all in one. Alternatively the chelator/EDTA can be introduced first to all daughter droplets and the detector introduced at different time points.

The screening and analysis can be performed in liquid reagent volumes, for example in microtitre plates or strip-tubes. Reagent volumes can be split such that portions are tested and portions retained for further use.

Such screening, characterising and purification can all be performed on a single device, which may be a digital microfluidic device. The term digital microfluidic device refers to a device having a two-dimensional array of planar microelectrodes. The term digital microfluidic device excludes any devices simply having droplets in a flow of oil in a channel. The droplets are moved over the surface by electrokinetic forces by activation of particular electrodes. Upon activation of the electrodes, the wettability of an aqueous droplet on the hydrophobic dielectric increases, thus causing the droplets to spread onto the surface. A digital microfluidic (DMF) device set-up is known in the art, and depends on the substrates used, the electrodes, the configuration of those electrodes, the use of a dielectric material, the thickness of that dielectric material, the hydrophobic layers, and the applied voltage.

The droplets can be aqueous droplets. The droplets can contain an oil immiscible organic solvent such as for example DMSO. The droplets can be a mixture of water and solvent, providing the droplets do not dissolve into the bulk oil.

Digital microfluidics (DMF) refers to a two-dimensional planar surface platform for lab-on-a-chip systems that is based upon the manipulation of microdroplets. Droplets can be dispensed, moved, stored, mixed, reacted, or analyzed on a platform with a set of insulated electrodes. Digital microfluidics can be used together with analytical analysis procedures such as mass spectrometry, colorimetry, electrochemical, and electrochemiluminescense.

The droplet can be moved using any means of electrokinesis. The aqueous droplet can be moved using electrowetting-on-dielectric (EWoD). Electrowetting on a dielectric (EWoD) is a variant of the electrowetting phenomenon that is based on dielectric materials. During EWoD, a droplet of a conducting liquid is placed on a dielectric layer with insulating and hydrophobic properties. Upon activation of the electrodes the wettability of an aqueous droplet on the hydrophobic dielectric increases, thus causing the droplet to spread onto the surface.

The electrical signal on the EWoD or optically-activated amorphous silicon (a-Si) EWoD device can be delivered through segmented electrodes, active-matrix thin-film transistors or digital micromirrors. Optically-activated s-Si EWoD devices are well known in the art for actuating droplets (J. Adhes. Sci. Technol., 2012, 26, 1747-1771).

A source of supplemental oxygen can be supplied to the droplets. For example droplets or gas bubbles containing gaseous or dissolved oxygen can be merged with the aqueous droplets during the protein expression. Alternatively the source of oxygen can be a molecular source which releases oxygen. Alternatively the droplets can be moved to an air/liquid boundary to enable increased diffusion of oxygen from a gaseous environment. Alternatively the oil can be oxygenated.

The droplet can be formed before entering the microfluidic device and flowed into the device. Alternatively the droplets can be merged on the device. Included is a method comprising merging a first droplet containing a nucleic acid template such as a plasmid with a second droplet containing a cell-free system having the components for protein expression to form the droplet.

The droplets can be actuated on a hydrophobic surface on the digital microfluidic device (ACS Nano 2018, 12, 6, 6050-6058). The hydrophobic surface can be a hydrophobic surface such as polytetrafluoroethylene (PTFE), Teflon AF (DuPont Inc), CYTOP (AGC Chemicals Inc), or FluoroPel (Cytonix LLC). The hydrophobic surface may be modified in such a way to reduce biofouling, especially biofouling resulting from exposure to CFPS reagents or nucleic acid reagents. The hydrophobic surface may also be superhydrophobic, such as NeverWet (NeverWet LLC) or Ultra-Ever Dry (Flotech Performance Systems Ltd). Superhydrophobic surfaces prevent biofouling compared with typical fluorocarbon-based hydrophobic surfaces. Superhydrophobic surfaces thus prolong the capability of digital microfluidic devices to move CFPS droplets and general solutions containing biopolymers (RSC Adv., 2017, 7, 49633-49648). The hydrophobic surface can also be a slippery liquid infused porous surface (SLIPS), which can be formed by infusing Krtox-103 oil (DuPont) with porous PTFE film (Lab Chip, 2019, 19, 2275).

For electrowetting on dielectrics (EWoD), the change in contact angle of reagent upon the application of electric potential is an inverse function of surface tension. Thus, for low voltage EWoD operations, reduction in surface tension is achieved by addition of surfactants to reagents, which for CFPS reactions means to the lysate and to the DNA. This results in a dilution of the lysate, and it has been seen, in experiments, that diluting the lysate results in a decrease in expression level of the protein of interest. Thus performing CFPS on DMF where the surfactants are added to the solutions being moved will necessarily result in a dilution of the lysate and thus a decrease in the level of protein expression. In addition to being a problem in its own right, this further complicates extrapolation of on-DMF results to in-tube predictions of protein yield. An additional detriment of having to add surfactants to the samples is that this increases the time required for sample preparation, as well as increasing the potential for inconsistent results due to ‘user error,’ as there is more handling of reagents. An additional detriment of having to add surfactants to the samples is that certain downstream operations are hindered. For example, if a protein of interest is expressed in a cell-free system with a GFP₁₁ (or similar) peptide tag, it's downstream complementation with a GFP₁₋₁₀ detector polypeptide is hindered in the presence of surfactant.

Rather than adding high levels of surfactants to the aqueous sample, it is instead possible to add surfactant, such as Span85 (sorbitan trioleate), to the oil. This has the advantages of enabling CFPS reactions to proceed on-DMF without dilution or adulteration. Additionally, it simplifies the sample preparation procedure for setting up the reactions, increasing the ease of use and the consistency of results. Using 1% w/w Span85 in dodecane allows for dilution-free CFPS reactions on-DMF, as well as dilution-free detection of the expressed non-fluorescent proteins. Other surfactants besides Span85, and oils other than dodecane could be used. A range of concentrations of Span85 could be used. Surfactants could be nonionic, anionic, cationic, amphoteric. Oils could be mineral oils or synthetic oils, including silicone oils, petroleum oils, and perfluorinated oils. Surfactants can have a detrimental effect on (1) the CFPS reactions and (2) the efficiency of the detection system (if the detection system involves complementation of a tag and detector). For example, by performing the CFPS reaction on-DMF with oil-surfactant mix, the detection of the expressed protein can also proceed without dilution and without adding aqueous surfactant. It has been shown that surfactants reduce the efficiency of some detection systems, including but not limited to the split GFP system, so removing surfactants from the reagent mix and instead adding them to the oil can be beneficial.

The filler liquid may be a hydrophobic or non-ionic liquid. For example the filler liquid may be decane or dodecane. The filler fluid may be a silicone oil such as dodecamethylpentasiloxane (DMPS). The filler liquid may contain a surfactant, for example a sorbitan ester such as Span 85.

Disclosed is a method of taking multiple droplets having a protein with a GFP₁₁ tag and monitoring changes in the level of the GFP₁₁ tag in order to determine protein stability. Disclosed is a method comprising expressing proteins in droplets on a digital microfluidic device having a two-dimensional array of planar microelectrodes wherein the proteins have a GFP₁₁ peptide amino sequence tag, holding the droplets at a fixed temperature and monitoring the droplets for changes in the level of the amount of the GFP₁₁ peptide amino sequence tag in the droplets.

Disclosed is a method comprising expressing one or more proteins in one or more droplets on a digital microfluidic device having a two-dimensional array of planar microelectrodes, wherein the proteins have a ccGFP₁₁ peptide amino sequence tag and a Strep-tag, splitting the droplets into at least two volumes, merging droplets containing ccGFP₁₋₁₀ into one of the droplets following the expression process and measuring the uniformity of the fluorescent signal to determine the degree of aggregation of the expressed protein, taking corresponding droplets without ccGFP₁₋₁₀ which contain protein having been solubly expressed and adding magnetic beads which bind to the strep-tag, immobilizing and washing the beads to remove unbound material; eluting the POI by disrupting the strep binding, adding droplets containing ccGFP₁₋₁₀ following the elution process and measuring the fluorescent signal in order to determine the level of protein purification and determining the conditions where purified soluble protein is obtained.

### Devices

The manipulation of droplets by the application of electrical potential can be achieved on electrodes covered with an insulator or a dielectric or a series of insulators or dielectrics. Droplet manipulation as a result of an applied electrical potential is known as electrowetting. Electrowetting occurs as result of a non-uniform electric field that influences the hydrostatic equilibrium of a dielectric liquid (dielectrophoresis or DEP) or a change in the contact angle of the liquid on solid surface (electrowetting-on-dielectric or EWoD). DEP can also be used to create forces on polarizable particles to induce their movement. The electrical signal can be transmitted to a discrete electrode, a transistor, an array of transistors, or a sheet of semi-conductor film whose electrical properties can be modulated by an optical signal.

EWoD phenomena occur when droplets are actuated between two parallel electrodes covered with a hydrophobic insulator or dielectric. The electric field at the electrode-electrolyte interface induces a change in the surface tension, which results in droplet motion as a result of a change in droplet contact angle. The electrowetting effect can be quantitatively treated using Young-Lippmann equation:

$$cos θ - cos {θ}_{0} = ( 1 / 2 γ LG ) c . {V}^{2}$$

where θ₀ is the contact angle when the electric field across the interfacial layer is zero, γLG is the liquid-gas tension, c is the specific capacitance (given as εᵣ. ε₀/t, where εᵣ is dielectric constant of the insulator/dielectric, ε₀ is permittivity of vacuum, t is thickness) and V is the applied voltage or electrical potential. The change in contact angle (inducing droplet movement) is thus a function of surface tension, electrical potential, dielectric thickness, and dielectric constant.

When a droplet is actuated by EWoD, there are two opposing sets of forces that act upon it: an electrowetting force induced by electric field and resistant forces that include the drag forces resulting from the interaction of the droplet with filler medium and the contact line friction (ref). The minimum voltage applied to balance the electrowetting force with the sum of all drag forces (threshold voltage) is variably determined by the thickness-to-dielectric contact ratio of the insulator/dielectric, (t/εᵣ)^{1/2}. Thus, to reduce actuation voltage, it is required to reduce (t/εᵣ)^{1/2} (i.e., increase dielectric constant or decrease insulator/dielectric thickness). To achieve low voltage actuation, thin insulator/dielectric layers must be used. However, the deposition of high quality thin insulator/dielectric layers is a technical challenge, and these thin layers are easily damaged before the desired electrowetting contact angle is large enough to drive the droplet is achieved. Most academic studies thus report the use of much higher voltages >100V on easily fabricated, thick dielectric films (>3 μm) to effect electrowetting.

High voltage EWoD-based devices with thick dielectric films, however, have limited industrial applicability largely due to their limited droplet multiplexing capability. The use of low voltage devices including thin-film transistors (TFT) and optically-activated amorphous silicon layers (a-Si) have paved the way for the industrial adoption of EWoD-based devices due to their greater flexibility in addressing electrical signals in a highly multiplex fashion. The driving voltage for TFTs or optically-activated a-Si are low (typically <15 V). The bottleneck for fabrication and thus adoption of low voltage devices has been the technical challenge of depositing high quality, thin film insulators/dielectrics. Hence there has been a particular need for improving the fabrication and composition of thin film insulator/dielectric devices.

Typically, the electrodes (or the array elements) used for EWoD are covered with (i) a hydrophilic insulator/dielectric and a hydrophobic coating or (ii) a hydrophobic insulator/dielectric. Commonly used hydrophobic coatings comprise of fluoropolymers such as Teflon AF 1600 or CYTOP. The thickness of this material as a hydrophobic coating on the dielectric is typically <100 nm and can have defects in the form of pinholes or a porous structure; hence, it is particularly important that the insulator/dielectric is pinhole free to avoid electrical shorting. Teflon has also been used as an insulator/dielectric, but it has higher voltage requirements due to its low dielectric constant and the thickness required to make it pinhole free. Other hydrophobic insulator/dielectric materials can include polymer-based dielectrics such as those based on siloxane, epoxy (e.g. SU-8), or parylene (e.g., parylene N, parylene C, parylene D, or parylene HT). Due to minimal contact angle hysteresis and a higher contact angle with aqueous solutions, Teflon is still used as a hydrophobic topcoat on these insulator/dielectric polymers. However, there are difficulties in reliably producing <1 micron pinhole-free coatings of parylene or SU-8; thus, the thickness of these materials is typically kept at 2-5 microns at the cost of increased voltage requirements for electrowetting. It has also been reported that traditional EWoD devices with parylene C are easily broken and unstable for repeated droplet manipulation with cell culture medium. Multi-layer insulator devices deposited with metal-oxide and parylene C films have been used to produce a more robust insulator/dielectric and enable operations with lower applied voltages. Inorganic materials, such metal oxides and semiconductor oxides, commonly used in the CMOS industry as “gate dielectrics”, have been used as insulator/dielectric for EWoD devices. They offer the advantage of utilizing standard cleanroom processes for thin film depositions (<100 nm). These materials are inherently hydrophilic, requiring an additional hydrophobic coating, and can be prone to pinhole formation as a result of thin film layer deposition process. Together with the need for lower voltage operations of EWoD, recent developmental work has focused on (1) using materials with improved dielectric properties (e.g., using high-dielectric constant insulators/dielectrics), (2) optimizing the fabrication process to make the insulator/dielectric pinhole free to avoid dielectric breakdown.

Operation of EWoD devices suffers from contact angle saturation and hysteresis, which is believed to be brought about by either one or combination of these phenomena: (1) entrapment of charges in the hydrophobic film or insulator/dielectric interface, (2) adsorption of ions, (3) thermodynamic contact angle instabilities, (4) dielectric breakdown of dielectric layer, (5) the electrode-electrode-insulator interface capacitance (arising from the double layer effect), and (6) fouling of the surface (such as by biomacromolecules). One of the adverse effects of this hysteresis is reduced operational lifetime of the EWoD-based device.

Contact angle hysteresis is believed to be a result of charge accumulation at the interface or within the hydrophobic insulator after several operations. The required actuation voltage increases due to this charging phenomenon resulting in eventual catastrophic dielectric breakdown. The most probable explanation is that pinholes at the insulator/dielectric may allow the liquid to come into contact with the electrode causing electrolysis. Electrolysis is further facilitated by pinhole-prone or porous hydrophobic insulators.

Most of the studies to understand contact angle hysteresis on EWoD have been conducted on short time scales and with low conductivity solutions. Long duration actuations (e.g., >1 hour) and high conductivity solutions (e.g., 1 M NaCl) could produce several effects other than electrolysis. The ions in solution can permeate through the hydrophobic coat (under the applied electric field) and interact with the underlying insulator/dielectric. Ion permeation can result in (1) change in dielectric constant due to charge entrapment (which is different from interfacial charging) and (2) change in surface potential of a pH sensitive metal oxide. Both can result in reduction of electrowetting forces to manipulate aqueous droplets, leading to contact angle hysteresis. The inventors have previously found that the damage from high conductivity solutions reduces or disables electrowetting on electrodes by inhibiting the modulation of contact angle when an electric field is applied.

An electrokinetic device includes a first substrate having a matrix of electrodes, wherein each of the matrix electrodes is coupled to a thin film transistor, and wherein the matrix electrodes are overcoated with a functional coating comprising: a dielectric layer in contact with the matrix electrodes, a conformal layer in contact with the dielectric layer, and a hydrophobic layer in contact with the conformal layer; a second substrate comprising a top electrode; a spacer disposed between the first substrate and the second substrate and defining an electrokinetic workspace; and a voltage source operatively coupled to the matrix electrodes.

The dielectric layer may comprise silicon dioxide, silicon oxynitride, silicon nitride, hafnium oxide, yttrium oxide, lanthanum oxide, titanium dioxide, aluminum oxide, tantalum oxide, hafnium silicate, zirconium oxide, zirconium silicate, barium titanate, lead zirconate titanate, strontium titanate, or barium strontium titanate. The dielectric layer may be between 10 nm and 100 μm thick. Combinations of more than one material may be used, and the dielectric layer may comprise more than one sublayer that may be of different materials.

The conformal layer may comprise a parylene, a siloxane, or an epoxy. It may be a thin protective parylene coating in between the insulating dielectric and the hydrophobic coating. Typically, parylene is used as a dielectric layer on simple devices. In this invention, the rationale for deposition of parylene is not to improve insulation/dielectric properties such as reduction in pinholes, but rather to act as a conformal layer between the dielectric and hydrophobic layers. The inventors find that parylene, as opposed to other similar insulating coatings of the same thickness such as PDMS (polydimethylsiloxane), prevent contact angle hysteresis caused by high conductivity solutions or solutions deviating from neutral pH for extended hours. The conformal layer may be between 10 nm and 100 μm thick. The conformal layer may be between 100 nm and 200 nm thick.

The hydrophobic layer may comprise a fluoropolymer coating, fluorinated silane coating, manganese oxide polystyrene nanocomposite, zinc oxide polystyrene nanocomposite, precipitated calcium carbonate, carbon nanotube structure, silica nanocoating, or slippery liquid-infused porous coating.

The elements may comprise one or more of a plurality of array elements, each element containing an element circuit; discrete electrodes; a thin film semiconductor in which the electrical properties can be modulated by incident light; and a thin film photoconductor whose properties can be modulated by incident light.

The functional coating may include a dielectric layer comprising silicon nitride, a conformal layer comprising parylene, and a hydrophobic layer comprising an amorphous fluoropolymer. This has been found to be a particularly advantageous combination.

The electrokinetic device may include a controller to regulate a voltage provided to the individual matrix electrodes. The electrokinetic device may include a plurality of scan lines and a plurality of gate lines, wherein each of the thin film transistors is coupled to a scan line and a gate line, and the plurality of gate lines are operatively connected to the controller. This allows all the individual elements to be individually controlled.

The second substrate may also comprise a second hydrophobic layer disposed on the second electrode. The first and second substrates may be disposed so that the hydrophobic layer and the second hydrophobic layer face each other, thereby defining the electrokinetic workspace between the hydrophobic layers.

The method is particularly suitable for aqueous droplets with a volume of 1 μL or smaller.

The EWoD-based devices shown and described below are active matrix thin film transistor devices containing a thin film dielectric coating with a Teflon hydrophobic top coat. These devices are based on devices described in the E Ink Corp patent filing on “Digital microfluidic devices including dual substrate with thin-film transistors and capacitive sensing”, US patent application no 2019/0111433, incorporated herein by reference.

Described herein are electrokinetic devices, including:
a first substrate having a matrix of electrodes, wherein each of the matrix electrodes is coupled to a thin film transistor, and wherein the matrix electrodes are overcoated with a functional coating comprising:
a dielectric layer in contact with the matrix electrodes,
a conformal layer in contact with the dielectric layer, and
a hydrophobic layer in contact with the conformal layer;
   a second substrate comprising a top electrode;
   a spacer disposed between the first substrate and the second substrate and defining an electrokinetic workspace; and
   a voltage source operatively coupled to the matrix electrodes;

Described herein is an electrokinetic device, including:
a first substrate having a matrix of electrodes, wherein each of the matrix electrodes is coupled to a thin film transistor, and wherein the matrix electrodes are overcoated with a functional coating comprising:
one or more dielectric layer(s) comprising silicon nitride, hafnium oxide or aluminum oxide in contact with the matrix electrodes,
a conformal layer comprising parylene in contact with the dielectric layer, and
a hydrophobic layer in contact with the conformal layer;
   a second substrate comprising a top electrode;
   a spacer disposed between the first substrate and the second substrate and defining an electrokinetic workspace; and
   a voltage source operatively coupled to the matrix electrodes;

The electrokinetic devices as described may be used with other elements, such as for example devices for heating and cooling the device or reagent cartridges for the introduction of reagents as needed.

“Droplet” refers to a volume of liquid that electrowets a hydrophobic surface and is at least partially bounded by carrier fluid and/or, in some instances, a gas or gaseous mixture such as ambient air. For example, a droplet may be completely surrounded by carrier fluid or may be bounded by carrier fluid and one or more surfaces of an EWoD device. Droplets may take a wide variety of shapes; non-limiting examples include generally disc shaped, slug shaped, truncated sphere, ellipsoid, spherical, partially compressed sphere, hemispherical, ovoid, cylindrical, and various shapes formed during droplet operations, such as merging or splitting or formed as a result of contact of such shapes with one or more working surface of an EWoD device. Droplets may include typical polar fluids such as water, as is the case for aqueous or non-aqueous compositions, or may be mixtures or emulsions including aqueous and non-aqueous components. Droplets may also include dispersions and suspensions, for example magnetic beads in an aqueous solvent. Droplets may also include one or more surfactants. In various embodiments, a droplet may include a biological sample, such as whole blood, lymphatic fluid, serum, plasma, sweat, tear, saliva, sputum, cerebrospinal fluid, amniotic fluid, seminal fluid, vaginal excretion, serous fluid, synovial fluid, pericardial fluid, peritoneal fluid, pleural fluid, transudates, exudates, cystic fluid, bile, urine, gastric fluid, intestinal fluid, fecal samples, liquids containing single or multiple cells, liquids containing organelles, fluidized tissues, fluidized organisms, liquids containing multi-celled organisms, biological swabs and biological washes.

“Droplet operation” refers to any manipulation of one or more droplets on a microfluidic device. A droplet operation may, for example, include: loading a droplet into the DMF device; dispensing one or more droplets from a source reservoir; splitting, separating or dividing a droplet into two or more droplets; moving a droplet from one location to another in any direction; merging or combining two or more droplets into a single droplet; diluting a droplet; mixing a droplet; agitating a droplet; deforming a droplet; holding a droplet in position; incubating a droplet; heating a droplet; vaporizing a droplet; cooling a droplet; disposing of a droplet; transporting a droplet out of a microfluidic device; other droplet operations described herein; and/or any combination of the foregoing. The terms “merge,” “merging,” “combine,” “combining” and the like are used to describe the creation of one droplet from two or more droplets. It should be understood that when such a term is used in reference to two or more droplets, any combination of droplet operations that are sufficient to result in the combination of the two or more droplets into one droplet may be used. For example, “merging droplet A with droplet B,” can be achieved by transporting droplet A into contact with a stationary droplet B, transporting droplet B into contact with a stationary droplet A, or transporting droplets A and B into contact with each other. The terms “splitting,” “separating” and “dividing” are not intended to imply any particular outcome with respect to volume of the resulting droplets (i.e., the volume of the resulting droplets can be the same or different) or number of resulting droplets (the number of resulting droplets may be 2, 3, 4, 5 or more). The term “mixing” refers to droplet operations which result in more homogenous distribution of one or more components within a droplet. Examples of “loading” droplet operations includes but is not limited to microdialysis loading, pressure assisted loading, robotic loading, passive loading, and pipette loading. Droplet operations may be electrode-mediated. In some cases, droplet operations are further facilitated by the use of hydrophilic and/or hydrophobic regions on surfaces and/or by physical obstacles.

For example 24 DNA constructs can be screened in 8 different conditions to determine the best construct and conditions for expression. The 24 constructs may be 24 independent sequences for different POIs or may be a single POI with 24 varying flanks (solubility tags etc) or a combination thereof (e.g 2 POI's with 12 flanks, 3 POI's with 8 flanks, 4 POI's with 6 flanks etc).

The 8 conditions may be lysates or reconstituted expression systems having different components or additives. 192 conditions may be screened in parallel. After identification of the best expression systems for a chosen POI, reagents/droplets can be selected for a purification screen. The screen may select for example 30 liquid volumes (droplets) to which beads are then added. The beads volumes may be made homogeneous by repeated merging with and splitting from other droplets having beads in order to homogenise the distribution of beads between droplets. The homogeneous bead droplets are then merged with droplets containing the expressed proteins chosen for the purification study. The beads may be washed one or more times to remove unbound material. The POI's may be eluted from the beads. Once detector reagent is added to the eluted POI's, soluble material can be measured. A lack of signal indicates either the POI did not bind to the bead or was not eluted from the bead. If the detectable signal is seen in clumps or patches, the eluted POI has aggregated during purification/elution and is not a good candidate for scale-up. The conditions where a high level of uniform signal is seen can be chosen for a subsequent scale-up using the same identified conditions for expression and purification. The conditions giving the best purification can be scaled up in tubes off device using the desired volumes of reagents to allow sufficient amounts of soluble proteins to be obtained. The level of signal measured by the purification screen allows the correct scale up volume to be identified to obtain the desired mass of the chosen protein. Such a scale up may allow for example greater than 20 g of protein in a 100 μL volume. At a suitable scale mg of protein may be obtained depending on the level of expression of the construct and the volume of solution used. The construct screen with different flank sequences allows placement of for example solubility tags at the C and/or N terminus along with varying configurations of the binding and detection tags, thereby increasing the likelihood that a particular POI can be expressed and purified in soluble form.

### Experimental & Results

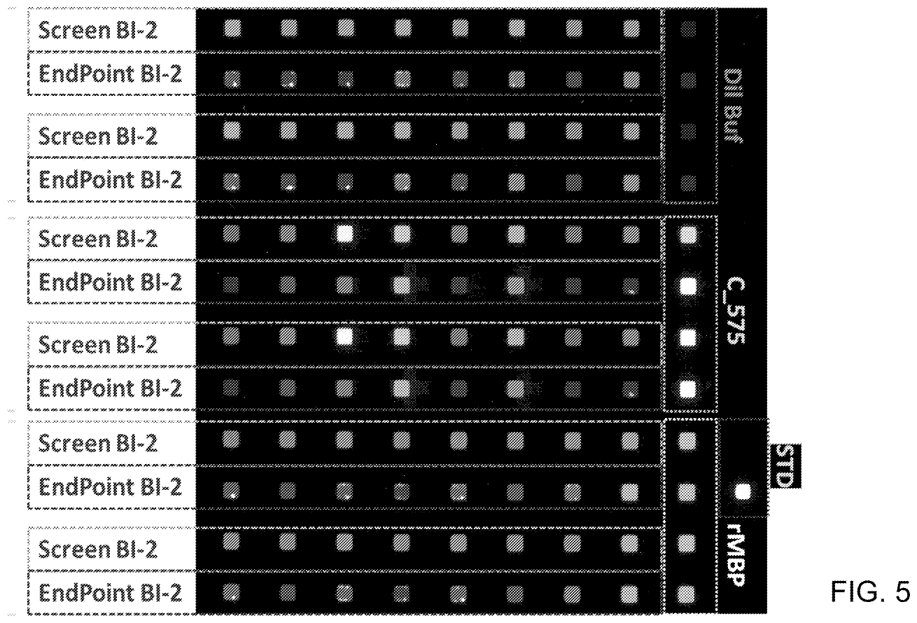

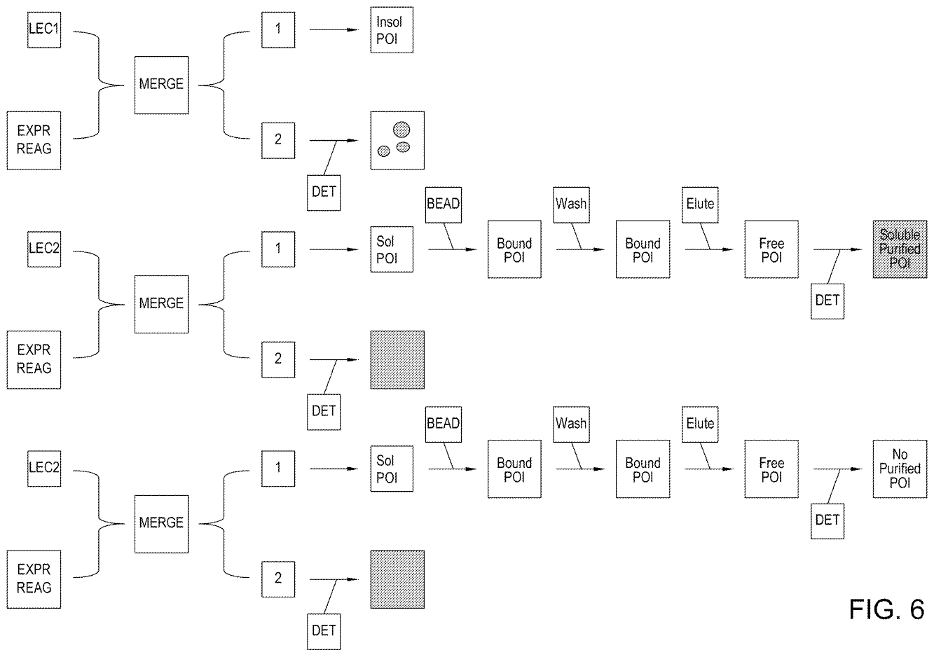

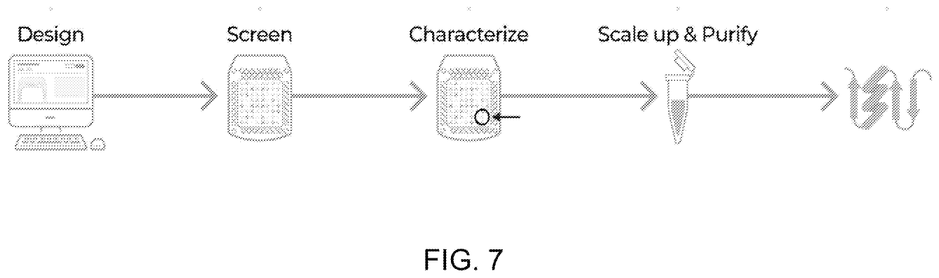

FIG. 5 shows the experimental result from 24 different proteins expressed in a reconstituted cell-free protein synthesis system in droplets on an electrowetting on dielectric (EWoD) device. Each construct contains a GFP₁₁ tag. In the rows marked Screen, the GFP₁₋₁₀ detector species is present from the start of expression. The rows marked Endpoint shows the fluorescence signal from 10 hours expression in the absence of GFP₁₋₁₀ detector species followed by 5 hours complementation with the GFP₁₋₁₀ detector species. This experiment showed significant differences between expression/complementation in Screen Biolnk compared to Endpoint detection. The detected protein clusters formed after expression only with endpoint detection mean that the protein aggregated after expression, thereby lowering the soluble yield. It is evident from the image the presence of speckles for several constructs, indicating the likely presence of protein aggregates. The level of aggregation enables identification of conditions which are worthy of further testing, and identification of conditions having high level of aggregated protein from which further purification is unlikely to give material.

### VEGF Expression

Human vascular endothelial growth factor (VEGF) is a key regulator of angiogenesis and plays a central role in the process of tumor growth and metastatic dissemination. VEGF also has involvement with neovascular age-related macular degeneration and rheumatoid arthritis.

VEGF protein has previously been recombinantly produced in vivo using yeast, insect and mammalian cell expression systems. Expressing this protein in E. coli has proven to be challenging due to the inherent cysteine-knot motif in human VEGF proteins and can result in protein misfolding and the formation of inclusion bodies. Expressing soluble VEGF in E. coli is only possible in the presence of solubility fusion tags such as maltose-binding protein (MBP). Moreover, MBP-VEGF proteins can only express successfully in E. coli Origami 2 (CE3) strains which carry specific gene mutations that facilitate proper disulfide bond formation. Optimizing protein expression in E. coli cells can be laborious and time consuming, taking days to weeks to discover the ideal condition to express and purify soluble proteins. E. coli protein expression involves cloning the protein-of-interest in various expression plasmids encoding different solubility tags, trying expression in different E. coli cell lines and optimizing different expression conditions. The inventors have produced VEGF in a microfluidic system using cell-free reagents derived from E. coli.

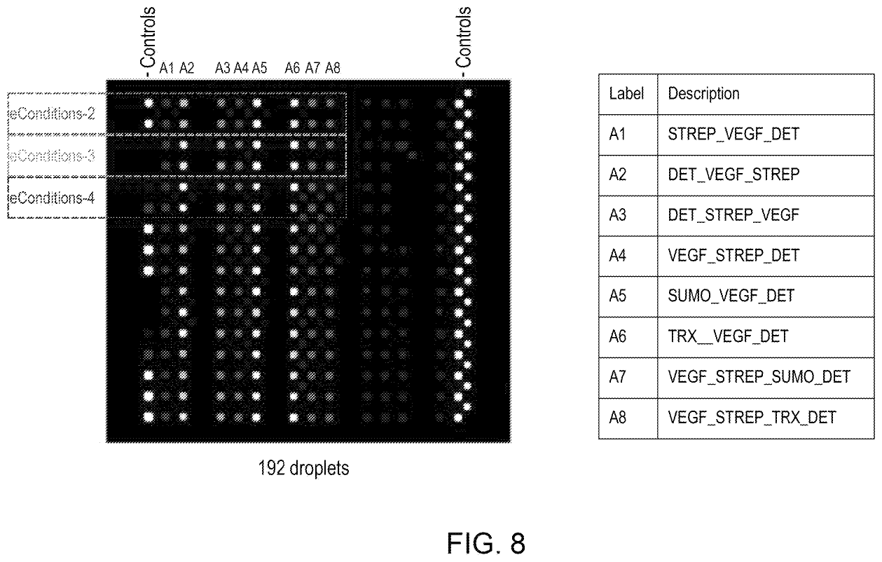

FIG. 8 shows an expression screen for protein VEGF with varying flank sequences. These 8 protein constructs were generated with different solubility tags attachment in either the N- or C-terminus. Strep II tag was included for purification purposes (Table 1):

| Label | Description |
|---|---|
| A1 | STREP_VEGF_DET |
| A2 | DET_VEGF_STREP |
| A3 | DET_STREP_VEGF |
| A4 | VEGF_STREP_DET |
| A5 | SUMO_VEGF_STREP_DET |
| A6 | TRX_VEGF_STREP_DET |
| A7 | VEGF_STREP_SUMO_DET |
| A8 | VEGF_STREP_TRX_DET |

Table 1. DNA Constructs Corresponding to VEGF with Various Solubility Tags in the N- and C-Terminus.

An expression and solubility screen was first performed to determine the optimal expression of human VEGF using three different cell-free reagents in combination with constructs containing 8 different nucleic acid template variations corresponding to VEGF with various solubility tags in the N- and C-terminus. These three cell-free blends consist of different cell-free protein synthesis reagents. Blend-3 is designed to promote disulfide bond formation while Blend-4 contains chaperones that promote protein folding. Blend-2 contains the core component of the cell-free synthesis reagents without any additional components. Each construct was run in duplicate. 192 droplets were run in total on a single cartridge. The dotted lines denote the cell-free blends run with the corresponding constructs. The position of the detector (DET) is also noted. Bright white fluorescent spots indicate expression of soluble proteins.

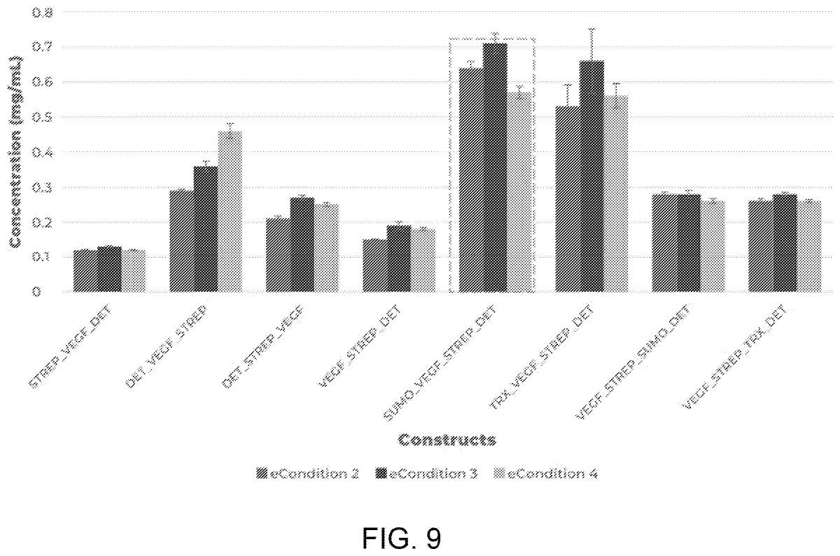

FIG. 9 shows a graphical representation of spot intensity in FIG. 8. Concentration of proteins expressed is calculated based on fluorescent intensity measured. Construct A5 (SUMO_VEGF_STREP_DET) produced the highest expression in Blend-3. Constructs with the highest yield (mg/mL) were selected for on-cartridge purification (highlighted in green dashed box).

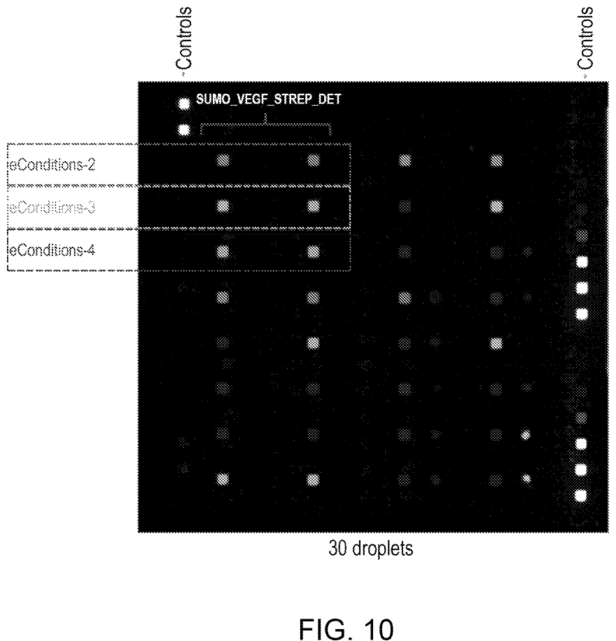

FIG. 10 shows an image of thirty soluble candidates that presented the highest expression yield which were automatically selected and taken forward for an on-cartridge purification step. Among these candidates were SUMO-VEGF expressed in Blend-2, Blend-3 and Blend-4. These expression candidates were purified on the cartridge using magnetic beads. FIG. 5 shows the location of SUMO_VEGF_STREP_DET purified on the cartridge. Fluorescent intensity measurement was used to determine yield of the purified proteins.

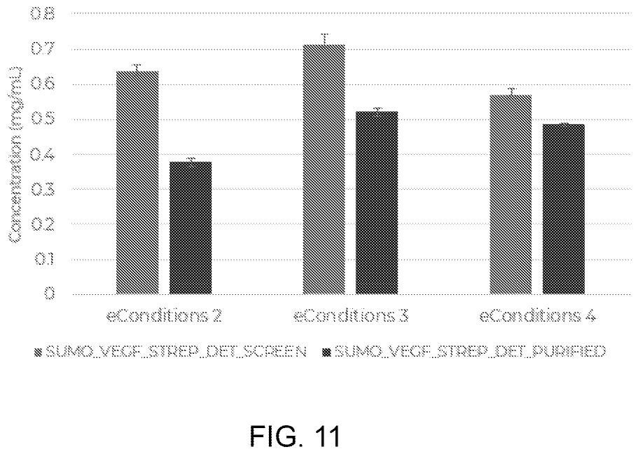

FIG. 11 shows a graphical representation of FIG. 10 and shows a side by side comparison of the expression yield and purified yield for SUMO-VEGF-STREP_DET. The results show that this construct, expressed in all three cell-free blends, is soluble and can be purified. Blend-3 yields the highest protein expression and purified yield and therefore was chosen for off-cartridge scale up expression.

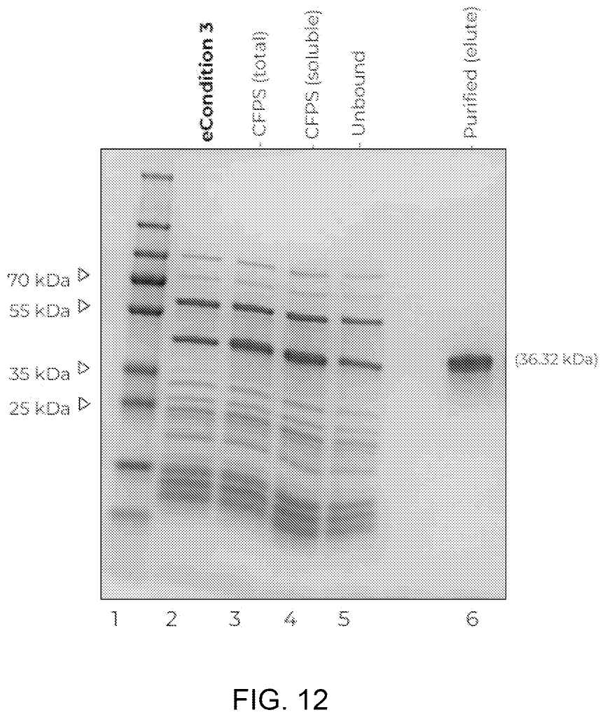

Linear DNA construct corresponding to SUMO_VEGF_STREP_DET was added to 500 μL of Blend-3 expression reagent in tubes and incubated overnight. Expressed proteins were purified using magnetic beads. Sumo-tagged VEGF proteins were eluted in 100 μL elution buffer and the eluted proteins were >95% pure as seen in FIG. 12 SDS-PAGE gel analysis. The final purified yield of SUMO_VEGF is 32.1 μg of proteins obtained. FIG. 12 shows a gel showing off cartridge, scaled-up expression (500 μL CFPS reaction) and purification of SUMO_VEGF_STREP_DET. SDS-PAGE illustrates the VEGF protein with a molecular weight slightly higher than the predictive weight of 36.32 kDa as the SUMO tag normally shows higher than its calculated molecular weight on an SDS-PAGE gel. Lane 1 molecular weight ladder, lane 2 Blend-3 negative control, lane 3 CFPS total after expression, lane 4 CPFS (soluble) obtained following centrifugation and soluble fraction obtained, lane 5 Unbound; flow through sample not bound to beads, lane 6 Purified (elute); sample eluted off of the resin bead.

### VEGF Protein Validation Data Summary and Conclusions

VEGF is a difficult candidate to express in the E. coli expression system. Expression of tag-less VEGF tends to end up with inclusion body formation. To obtain soluble proteins, expressing VEGF with multiple combinations of solubility tags is required to determine the right construct for successful expression and purification. The conventional cell-based expression and purification approaches can be time consuming and laborious, considering the variety of constructs that need to be tested. We have demonstrated that a droplet microfluidic platform enables rapid construct expression screening and selection of soluble, purifiable candidates. VEGF with N-terminus SUMO tag gave the highest expression yield in Blend-3 expression reagent. Results generated on the platform were validated through an off-cartridge cell-free expression. The expressed proteins were purified to high purity and yield; comparable to VEGF proteins produced in cell-based expression platforms.

Bead Homogenisation of an 8×4 Array with Droplet Repositioning Following Each Cycle

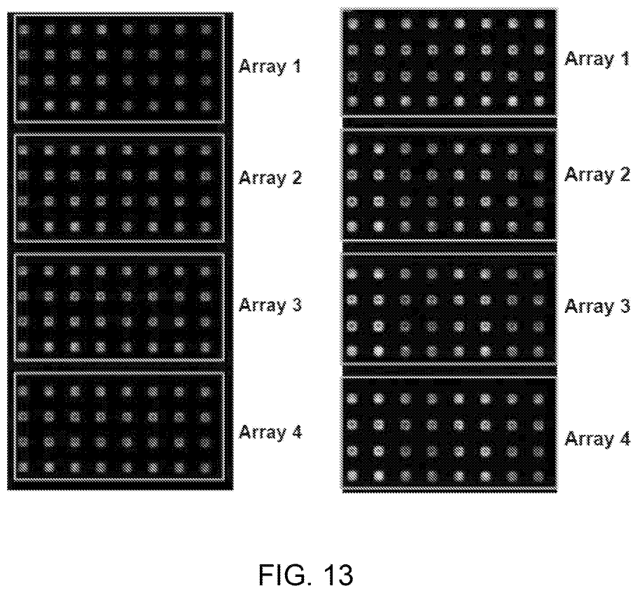

Magnetic particles were visualized and quantified via fluorescent imaging of fluorescein bound to the surface of the magnetic particle. An array of 32× droplets containing 25 μm Streptactin coated magnetic microparticles were dispensed in 49 px² droplets in an 8×4 formation. FIG. 13 shows a repeat of four arrays across 2 devices.

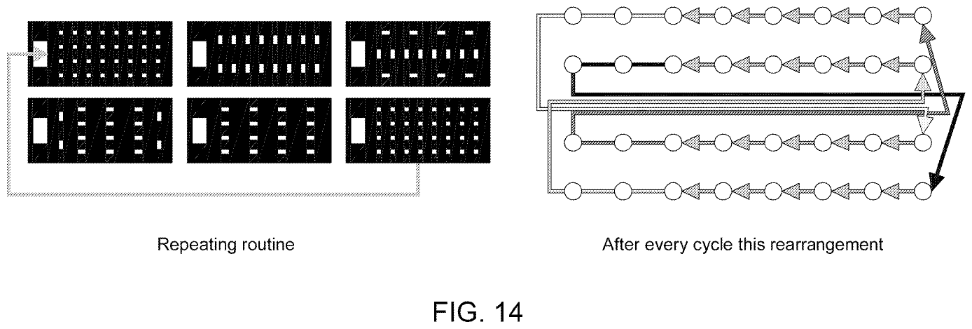

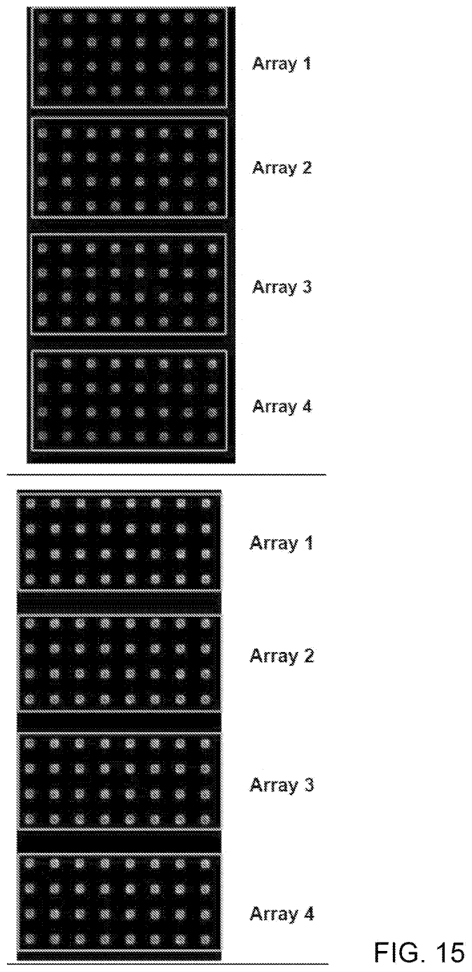

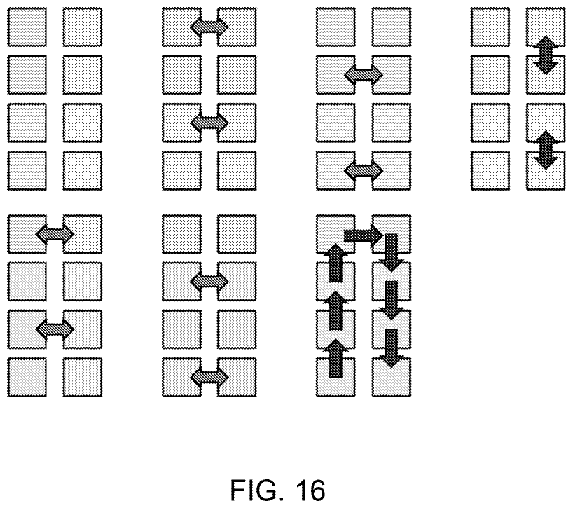

Each array undergoes a routine as described below for a number of cycles (40 in this case) as shown in FIG. 14, where magnetic bead containing droplets are mixed and split repeatedly. A fluorescent image of the arrays following the cycling is shown in FIG. 15. A comparison of images 13 and 15 shows that for the starting images in FIG. 13 there is a wide difference between the brightest and darkest droplets, indicating a wide disparity in bead concentrations. FIG. 15 shows a lower level variation between the droplet brightness, indicating the level of beads has been made homogeneous.

Analysis of the intensity of the fluorescent signal from each droplet was performed to give a comparison of the bead mass per droplet from the images above both before and after the cycling:

| | |
|---|---|
| Data set 1 | Data set 2 |

| | % CV | % CV | % CV | % CV |
|---|---|---|---|---|
| | initial | after 40X | initial | after 40X |
| Reservoir | array | cycles | array | cycles |
| C1 | 26 | 9 | 13 | 6 |
| C3 | 12 | 8 | 23 | 5 |
| C5 | 11 | 10 | 27 | 6 |
| C7 | 13 | 12 | 25 | 9 |

The data shows the bead concentrations can be normalised by repeatedly combining and splitting the droplets, then moving to the next droplets and combining and splitting. Each droplet is mixed with at least one other droplet in order to spread the beads between droplets and overcome any initial errors caused by dispensed bead volumes.

### Bead Homogenisation Measured by the Pelleting and Resuspending of Homogenized Droplets.

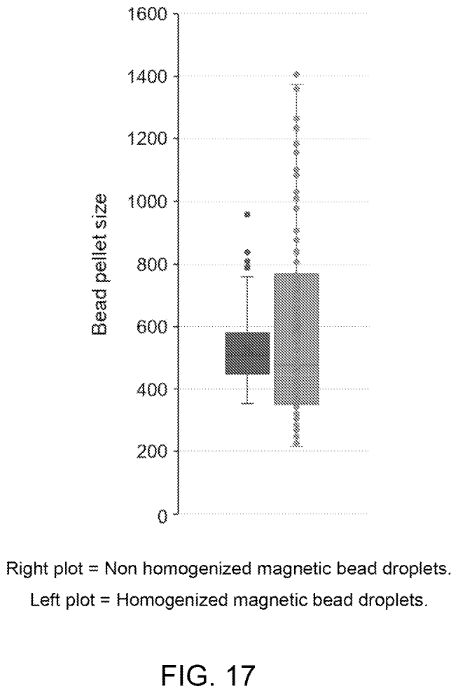

The 4×8 array homogenisation as described above was performed on the same cartridge as an array of beads which did not undergo the homogenisation and this was performed across 4X cartridges, with 32 droplets per cartridge which underwent homogenisation and 32 droplets which did not. The unmixed mixed beads lost 21.1% of the bead pellets (27 out of 128) while the homogenised/mixed beads lost <1% of bead pellets (1 out of 128). The pelleted size distribution is shown in FIG. 17. The beads are uniformly mixed across the various droplets, and the number of droplets where the beads are completely lost is greatly reduced.

1. A method for creating aqueous droplets having homogeneous concentrations of suspended beads, wherein the method comprises:
a. taking a reservoir containing an aqueous liquid having beads suspended therein,
b. forming multiple droplet sub-volumes from the reservoir,
c. merging two or more of the droplets,
d. splitting the merged droplets,
e. rearranging the set of droplets, and
f. repeating steps c., d. and e. one or more times such that each of the final split droplets contains a homogeneous concentration of suspended beads.

2. The method according to claim 1, wherein the beads are magnetic.

3. The method according to claim 1 or claim 2, wherein each droplet formed from the reservoir merges with and splits from every other droplet formed from the reservoir.

4. The method according to any one of claims 1 to 3, wherein 8 or more droplets are produced.

5. The method according to any one of claims 1 to 3, wherein 32 or more droplets are produced.

6. The method according to any one of claims 1 to 5, wherein the method is performed in droplets on a digital microfluidic device.

7. The method according to claim 6, wherein the digital microfluidic device comprises active-matrix thin-film transistors.

8. The method according to any one preceding claim, wherein the beads are greater than 10 μm in diameter.

9. The method according to any one preceding claim, wherein the beads average 25 μm in diameter.

10. The method according to any one preceding claim wherein, the homogeneous bead droplets are merged with droplets having substances which bind to the beads.

11. The method according to any one preceding claim wherein the beads are used to bind to proteins.

12. A method for synthesising, characterising and purifying one or more proteins having detection tags and binding tags, the method comprising the steps of:
i mixing a cell-free protein expression system with one or more nucleic acid templates to form a plurality of merged liquid volumes; either
iia splitting the merged volumes into at least two aliquots and allowing protein expression in at least two aliquots; or
iib allowing protein expression then splitting into at least two aliquots;
iii adding a detector reagent which binds to the detection tags to at least one of the aliquots in order to determine the level of protein expression and determining the conditions where protein is expressed in soluble form;
iv selecting the volumes where protein has been solubly expressed and adding magnetic beads to a sub-population of the aliquots, wherein the beads bind to the binding tags and the beads in the droplets have been merged and split one or more times such that each of the final split droplets contains a homogeneous concentration of suspended beads;
v separating the magnetic beads from the expression reagents;
vi optionally adding wash reagents to the magnetic beads to remove unbound material;
vii eluting the bound protein from the magnetic beads by disrupting the binding to the binding tag; and
viii adding a detector reagent which binds to the detection tags to at least one of the eluted aliquots in order to determine the level of protein purification and determining the conditions where purified soluble protein is obtained.

13. The method according to claim 12, wherein the ratio of soluble and insoluble POI is determined in volumes where the detector reagent is added.

14. The method according to any one of claim 12 or 13, wherein each of the binding sequences contains four or more amino acids.

15. The method according to claim 14, wherein the detector reagent comprises a component of a fluorescent protein.

16. The method according to claim 15, wherein the protein of interest has a ccGFP₁₁ tag and the detector reagent comprises ccGFP₁₋₁₀.

17. The method according to any one of claims 12 to 16, wherein the POI is expressed with a solubility enhancer selected from:

| | |
|---|---|
| Glutathione S-Transferase | GST |
| Small Ubiquitin-like Modifier | SUMO |
| Maltose Binding Protein | MBP |
| Fasciola hepatica 8 kDa antigen | FH8 |
| Thioredoxin | TRX |
| Solubility Enhancing Ubiquitous Tag | SNUT |
| Seventeen kilodalton protein | SKP |
| Monomeric bacteriophage T7 orc protein | MOCR |
| E coli secreted protein A | ESPA |
| N-utilization substance | NusA |
| IgG domain B1 of Protein G | GB1 |
| IgG repeat domain ZZ of Protein A | ZZ |
| Mutated dehalogenase | HaloTag |
| Phage T7 protein kinase | T7PK |
| E. coli trypsin inhibitor | Ecotin |
| Calcium-binding protein | CaBP |
| Stress-response arsenate reductase | ArsC |
| N-terminal fragment of translation initiation factor IF2 | IF2-domain 1 |
| Stress-response protein | RpoA |
| Stress-response protein | SlyD |
| Stress-response protein | Tsf |
| Stress-response protein | RpoS |
| Stress-response protein | PotD |
| Stress-response protein | Crr |
| E. coli acidic protein | msyB |
| E. coli acidic protein | yjgD |
| E. coli acidic protein | rpoD |
| T7 phage tail | P17 |
| metal-binding protein | CUSF |
| 53-amino-acid-long N-terminal extension sequence | NEXT |

18. The method according to any one of claims 12 to 17, wherein the expression is performed using cell-free lysates or reconstituted expression systems.

19. The method according to any one of claims 1 to 18, wherein the digital microfluidic device comprises an oil-filled or humidified gaseous environment, wherein the humidified gaseous environment is achieved by enclosing or sealing the digital microfluidic device and providing on-board reagent reservoirs.

20. The method according to any one of claims 1 to 19, wherein the beads bind to a binding moiety selected from:
Alfa-tag
(SRLEEELRRRLTE)
Avi-tag
(GLNDIFEAQKIEWHE)
C-tag
(EPEA)
Calmodulin-tag
(KRRWKKNFIAVSAANRFKKISSSGAL)
Dogtag
(DIPATYEFTDGKHYITNEPIPPK)
E-tag
(GAPVPYPDPLEPR)
FLAG
(DYKDDDDK)
G4T
(EELLSKNYHLENEVARLKK)
HA
(YPYDVPDYA)
His
(HHHHHH)
Isopeptag
(TDKDMTITFTNKKDAE)
lanthanide binding tag (LBT)
(FIDTNNDGWIEGDELLLEEG)
Myc
(EQKLISEEDL)
NE-Tag
(TKENPRSNQEESYDDNES)
Poly Glutamate-tag
(EEEEEEE)
Poly Arginine-tag
(RRRRRRR)
Rho1D4-tag
(TETSQVAPA)
SBP-tag
(MDEKTTGWRGGHVVEGLAGELEQLRARLEHHPQGQREP)
Sdytag
(DPIVMIDNDKPIT)
SH3
(STVPVAPPRRRRG)
SNAC
(GSHHW)
Snooptag
(KLGDIEFIKVNK)
Softag 1
(SLAELLNAGLGGS)
Softag 3
(TQDPSRVG)
Spot-tag
(PDRVRAVSHWSS)
Spytag
(AHIVMVDAYKPTK)
S-tag
(KETAAAKFERQHMDS)
Strep-tag
(AWAHPQPGG)
(AWRHPQFGG)
Strep-tag II
(WSHPQFEK)
T7tag
(MASMTGGQQMG)
TC-tag
(EVHTNQDPLD)
Ty-tag
(CCPGCC)
VSV-tag
(YTDIEMNRLGK)
Xpress-tag
(DLYDDDDK)

21. The method according to any one preceding claim comprising expressing one or more proteins in one or more droplets on a digital microfluidic device having a two-dimensional array of planar microelectrodes, wherein the proteins have a ccGFP₁₁ peptide amino sequence tag and a Strep-tag, splitting the droplets into at least two volumes, merging droplets containing ccGFP₁₋₁₀ into one of the droplets following the expression process and measuring the uniformity of the fluorescent signal to determine the degree of aggregation of the expressed protein, taking corresponding droplets without ccGFP₁₋₁₀ which contain protein having been solubly expressed and adding magnetic beads which bind to the strep-tag, wherein the droplets having the magnetic beads have been merged and split one or more times such that each of the final split droplets contains a homogeneous concentration of suspended beads, immobilizing and washing the beads to remove unbound material; eluting the POI by disrupting the strep binding, adding droplets containing ccGFP₁₋₁₀ following the elution process and measuring the fluorescent signal in order to determine the level of protein purification and determining the conditions where purified soluble protein is obtained.

22. The method according to any one preceding claim, wherein at least 8 different nucleic acid templates are screened against at least 4 different expression reagents on the same device.

23. The method according to any one preceding claim, wherein the device can separately manipulate at least 32 droplets containing beads.

24. The method according to any one preceding claim, wherein the final dispensed homogenised concentration of the beads is 20% v/v+/−10% of each droplet.
